(19) United States
(12) Patent Application Publication
(10) Pub. No.: US 2026/0174438 A1
(43) Pub. Date: Jun. 25, 2026

(54) Release Mechanism for LAA Occluder with Electrodes

(71) Applicant: St. Jude Medical, Cardiology Division, Inc., St. Paul, MN (US)

(72) Inventors: Louis-Philippe Richer, Montreal (CA); Saurabh Datta, Pleasanton, CA (US)

(73) Assignee: St. Jude Medical, Cardiology Division, Inc., St. Paul, MN (US)

(21) Appl. No.: 19/339,542

(22) Filed: Sep. 25, 2025

Related U.S. Application Data
(60) Provisional application No. 63/737,217, filed on Dec. 20, 2024.

(51) Int. Cl. A61B 17/12; A61B 17/00

(52) U.S. Cl. CPC A61B 17/12172; A61B 17/12122; A61B 2017/00044; A61B 2017/00477; A61B 2017/12095

(57) ABSTRACT

A system for occluding a left atrial appendage (“LAA”) includes a delivery device and an LAA occluder. The LAA occluder has an indicator electrode coupled to a connector electrode via a conducting wire. The delivery device has a connector with a connector electrode configured to be coupled to a navigation system via a conducting wire. During implantation, the delivery device is coupled to the LAA occluder so that the connector electrodes are electrically coupled, resulting in the indicator electrode being conductively coupled to the navigation system. After the LAA occluder is deployed into the LAA, the delivery device is disconnected from the LAA occluder to allow the LAA occluder to remain in the patient.

### CROSS-REFERENCE TO RELATED APPLICATIONS

This application claims priority to the filing date of U.S. Provisional Patent Application No. 63/737,217, filed December 20, 2024, the disclosure of which is hereby incorporated by reference herein.

### BACKGROUND OF THE DISCLOSURE

The left atrial appendage (LAA) is a muscular pouch extending from the anterolateral wall of the left atrium of the heart. The LAA serves as a reservoir for the left atrium. During a normal cardiac cycle, the LAA contracts with the left atrium to pump blood to the left ventricle. This atrial contraction generally prevents blood from stagnating within the LAA. However, during cardiac cycles characterized by arrhythmias (e.g., atrial fibrillation), the LAA may fail to adequately contract. As a result, blood may stagnate within the LAA. Stagnant blood within the LAA is susceptible to coagulating and forming a thrombus, which can dislodge from the LAA and ultimately result in an embolic stroke.

Atrial fibrillation is among the most prevalent arrhythmias affecting more than 35 million people in the world. The main risk linked to atrial fibrillation is vascular cerebral stroke caused by blood clots created in cardiac chambers. The first line of treatment against blood clots remains anticoagulant drugs. However, long-term oral anticoagulation is contraindicated for some patients.

Another treatment is closure of the left atrial appendage. Typically, an LAA occlusion procedure is performed via a transseptal approach that requires both fluoroscopy and direct intravenous injection of iodine-based contrast in the left atrium. Under fluoroscopy, contrast injection is typically required to assess the geometry of the LAA, ensure appropriate positioning of the LAA occlusion device, and verify that occlusion of the LAA is achieved. However, contrast may be a nephrotoxin that is harmful to the kidneys. Furthermore, x-rays used in fluoroscopy are associated with potential harm to both patients and operating room personnel exposed to the x-rays.

Accordingly, it would be advantageous to reduce the dependence on fluoroscopy, including fluoroscopy with contrast, during intravascular procedures, including LAA occlusion procedures. However, at least some systems which can reduce the dependence on fluoroscopy can introduce new problems, such as a suitable way to detach the LAA occluder from a the delivery system used to implant the occluder.

### BRIEF SUMMARY OF THE DISCLOSURE

According to one aspect of the disclosure, a system for occluding a left atrial appendage (“LAA”) of a patient includes a delivery device including a catheter and a delivery cable configured to be received within the catheter, the delivery cable including a distal connector, the distal connector including a first distal connector electrode, a first delivery device conducting wire having a distal end coupled to the first distal connector electrode and having a proximal end configured to couple to a navigation system external to the patient. The system includes a collapsible and expandable LAA occluder having a first implant electrode, the first implant electrode being positioned on the LAA occluder so that, when the LAA occluder is in an expanded condition within the LAA of the patient, the first implant electrode is in contact with tissue of the LAA, the LAA occluder including a proximal connector, the proximal connector including a first proximal connector electrode, a first implant conducting wire having a distal end coupled to the first implant electrode and having a proximal end coupled to the first proximal connector electrode. The system has (i) a delivery condition in which the distal connector is connected to the proximal connector to couple the LAA occluder to the delivery cable so that the first distal connector electrode is in contact with the first proximal connector electrode to conductively couple the first implant electrode with the proximal end of the delivery device conducting wire via the first implant conducting wire, and (ii) an implanted condition in which the distal connector is disconnected from the proximal connector to decouple the LAA occluder from the delivery cable, so that the first distal connector electrode is not in contact with the first proximal connector electrode. In the expanded condition of the LAA occluder, the LAA occluder may include a distal lobe having a distal diameter, a proximal disc having a proximal diameter, and a waist connecting the proximal disc to the distal lobe, the waist having a waist diameter, the proximal diameter being larger than the distal diameter, the distal diameter being greater than the waist diameter. The proximal connector may include a tube portion, the tube portion extending into an interior of the disc and at least partially into an interior of the waist. The distal connector may have threads, and the proximal connector may have threads that are complementary to the threads of the distal connector. The system may be configured to transition from the delivery condition to the implanted condition via rotation of the distal connector relative to the proximal connector. The system may be configured to transition from the delivery condition to the implanted condition via pulling the distal connector proximally relative to the proximal connector. The first distal connector electrode may be retractable at least partially into an interior of the distal connector. The first distal connector electrode may be configured to retract at least partially into the interior of the distal connector by applying a proximal force on the first delivery device conducting wire. The distal connector may include an interior lumen, and the delivery cable may include an interior lumen, the first delivery device conducting wire extending through the interior lumen of the distal connector and through the interior lumen of the delivery cable. The occlusion system may also include the navigation system, and in the delivery condition of the system, the navigation system may be configured to receive signals from the first implant electrode via the first implant conducting wire and via the first delivery device conducting wire.

In some examples, (i) the first distal connector electrode is one of a plurality of distal connector electrodes, (ii) the first delivery device conducting wire is one of a plurality of distal conducting wires, each of the plurality of distal conducting wires being coupled to a respective one of the plurality of distal connector electrodes, (iii) the first implant electrode is one of a plurality of implant electrodes, (iv) the first proximal connector electrode is one of a plurality of proximal connector electrodes, and (v) the first implant conducting wire is one of a plurality of implant conducting wires, each of the plurality of implant electrodes being coupled to a respective one of the plurality of proximal connector electrodes via a respective one of the plurality of implant conducting wires. The plurality of distal connector electrodes may be arranged in a linear array, and the plurality of proximal electrode connectors may be arranged in a linear array. The plurality of distal connector electrodes may be arranged in a helical array, and the plurality of proximal electrode connectors may be arranged in a helical array. The plurality of implant electrodes may include (i) an indicator electrode configured to contact tissue when the LAA occluder is in the expanded condition within the LAA of the patient, the first implant electrode being the indicator electrode, and (ii) a reference electrode configured to not contact tissue when the LAA occluder is in the expanded condition within the LAA of the patient. The plurality of implant electrodes may include a second implant electrode, the first implant electrode being positioned on a proximal disc of the LAA occluder, the second implant electrode being positioned on a distal lobe of the LAA occluder.

According to another aspect of the disclosure, a method of occluding a left atrial appendage (“LAA”) of a patient includes advancing a LAA occluder through a catheter of a delivery device while the LAA occluder is in a collapsed condition and while a proximal connector of the LAA occluder is connected to a distal connector of a delivery cable of the delivery device in a delivery configuration. The method includes allowing the LAA occluder to expand within the LAA of the patient so that a first implant electrode on the LAA occluder contacts tissue of the LAA while the proximal connector remains connected to the distal connector. The method also includes receiving a signal transmitted from the first implant electrode to a navigation system external to the patient when the first implant electrode is in contact with tissue of the LAA, the signal being transmitted (i) from the first implant electrode to a first proximal connector electrode on the proximal connector via a first implant conducting wire having a distal end coupled to the first implant electrode and having a proximal end coupled to the first proximal connector electrode, (ii) from the first proximal connector electrode to a first distal connector electrode on the distal connector, and (iii) from the first distal connector electrode to the navigation system via a first delivery device conducting wire having a distal end coupled to the first distal connector electrode and having a proximal end coupled to the navigation system. After receiving the signal, the distal connector is disconnected from the proximal connector so that the LAA occluder disconnects from the delivery device and so that the first distal connector electrode is no longer in contact with the first proximal connector electrode. After receiving the signal but before disconnecting the distal connector from the proximal connector, contact between the LAA occluder and the tissue of the LAA may be evaluated based on the signal received by the navigation system. In some examples, (i) the first distal connector electrode is one of a plurality of distal connector electrodes, (ii) the first delivery device conducting wire is one of a plurality of distal conducting wires, each of the plurality of distal conducting wires being coupled to a respective one of the plurality of distal connector electrodes, (iii) the first implant electrode is one of a plurality of implant electrodes, (iv) the first proximal connector electrode is one of a plurality of proximal connector electrodes, and (v) the first implant conducting wire being is of a plurality of implant conducting wires, each of the plurality of implant electrodes being coupled to a respective one of the plurality of proximal connector electrodes via a respective one of the plurality of implant conducting wires. Upon allowing the LAA occluder to expand within the LAA of the patient, (i) the first implant electrode may be on a distal lobe of the LAA occluder and may contact tissue of an interior wall of the LAA, and (ii) a second implant electrode of the plurality of implant electrodes may be on a proximal disc of the LAA occluder and may contact tissue forming an ostium leading into the LAA. Upon allowing the LAA occluder to expand within the LAA of the patient, a third implant electrode of the plurality of implant electrodes may not contact tissue of the LAA, the third implant electrode being a reference electrode, the first and second implant electrodes being indicator electrodes.

According to another aspect of the disclosure, an implantation system includes a delivery device including a catheter including a distal connector, the distal connector including a first distal connector electrode, a first delivery device conducting wire having a distal end coupled to the first distal connector electrode and having a proximal end configured to couple to a navigation system external to the patient. The system includes a prosthetic implant configured to be implanted into a heart of a patient, the prosthetic implant having a first implant electrode, the first implant electrode being positioned on the prosthetic implant so that, when the prosthetic implant is implanted into the heart of the patient, the first implant electrode is in contact with tissue of the heart, the prosthetic implant including a proximal connector, the proximal connector including a first proximal connector electrode, a first implant conducting wire having a distal end coupled to the first implant electrode and having a proximal end coupled to the first proximal connector electrode. The system has (i) a delivery condition in which the distal connector is connected to the proximal connector to couple the prosthetic implant to the delivery device so that the first distal connector electrode is in contact with the first proximal connector electrode to conductively couple the first implant electrode with the proximal end of the delivery device conducting wire via the first implant conducting wire, and (ii) an implanted condition in which the distal connector is disconnected from the proximal connector to decouple the prosthetic implant from the delivery device, so that the first distal connector electrode is not in contact with the first proximal connector electrode. The prosthetic implant may be a collapsible and expandable left atrial appendage (“LAA”) occluder configured to occlude a LAA of the patient. The prosthetic implant may be a collapsible and expandable prosthetic heart valve configured to replace a native heart valve of the patient. The prosthetic implant may be a transcatheter edge-to-edge (“TEER”) fixation device configured to couple a first native leaflet of a native heart valve of the patient to a second native leaflet of the native heart valve of the patient.

### BRIEF DESCRIPTION OF THE DRAWINGS

FIG. 1 is a schematic cutaway view of a human heart showing an example transapical delivery approach and an example transseptal delivery approach.

FIG. 2 is a schematic representation of the mitral valve, left atrial appendage, and associated structures during normal operation of the heart.

FIGS. 3A-3C illustrate an example LAA occlusion device.

FIGS. 4A-4B illustrate an example delivery device.

FIG. 5 is a schematic diagram of an example medical navigation system.

FIGS. 6A-6F illustrate stages of example LAA occlusion procedures.

FIGS. 7A-7D illustrate an example set of electrodes disposed on the surface of an example LAA occlusion device and example impedance data corresponding thereto.

FIG. 8A illustrates an example of an LAA occluder immediately after deployment into an LAA but prior to disconnection from the delivery system.

FIG. 8B is similar to FIG. 8B, showing the LAA occluder immediately after disconnection from the delivery system.

FIG. 9A is a schematic view of the LAA occluder of FIGS. 7A and 8A.

FIG. 9B is a schematic view of a connector of the LAA occluder of FIG. 9A.

FIG. 9C is a schematic view of a connector of the delivery device shown in FIGS. 8A-8B.

FIGS. 10A-B are schematic illustrations of ways in which the connector of FIG. 9B may connect to the connector of FIG. 9C.

FIG. 10C is a schematic illustration of the connectors of FIGS. 9B and 9C in a connected state.

FIG. 11 is a schematic illustration of the LAA occluder of FIG. 9A that illustrates exemplary connections between electrodes on the LAA occluder to electrodes on the connector of the LAA occluder.

FIG. 12 is a schematic illustration of the LAA occluder of FIG. 11 coupled to the navigation system of FIG. 5 via connection to the delivery system 70 of FIGS. 4A-B and 9C.

FIG. 13A is a cutaway view of a distal end portion of a delivery cable according to another aspect of the disclosure.

FIG. 13B is a perspective view of a distal end portion of a hollow connector shaft.

FIGS. 13C-E illustrate three stage of disconnecting the hollow connector shaft of FIG. 13B from the delivery cable of FIG. 13A.

FIG. 13F is a highly schematic view of a the delivery cable and hollow connector shaft of FIGS. 13A-B in an assembled and electrically connected condition with an LAA occluder.

FIG. 13G is a highly schematic view of the delivery cable of FIG. 13F still connected to the LAA occluder, but the hollow connector shaft disconnected and in an electrically disconnected condition.

FIG. 14 is a block diagram that illustrates a computer system upon which one or more examples may be implemented.

### DETAILED DESCRIPTION OF THE DISCLOSURE

In the following description, numerous specific details are set forth in order to provide a thorough understanding of the disclosure. It will be apparent, however, that the embodiments may be practiced without these specific details. The detailed description that follows describes exemplary embodiments and the features disclosed are not intended to be limited to the expressly disclosed combination(s). Therefore, unless otherwise noted, features disclosed herein may be combined to form additional combinations that were not otherwise shown for purposes of brevity.

As used herein, the term “proximal,” when used in connection with a delivery device or components of a delivery device, refers to the end of the device closer to the user of the device when the device is being used as intended. On the other hand, the term “distal,” when used in connection with a delivery device or components of a delivery device, refers to the end of the device farther away from the user when the device is being used as intended. In some instances, the terms “proximal” and “distal” may be arbitrarily assigned to facilitate understanding of the disclosure, and such instances will be readily apparent to the skilled artisan. As used herein, the terms “substantially,” “generally,” “approximately,” and “about” are intended to mean that slight deviations from absolute are included within the scope of the term so modified.

It will be further understood that: the term “or” may be inclusive or exclusive unless expressly stated otherwise; the term “set” may comprise zero, one, or two or more elements; the terms “first”, “second”, “certain”, and “particular” are used as naming conventions to distinguish elements from each other, and do not imply an ordering, timing, or any other characteristic of the referenced items unless otherwise specified; the term “and/or” as used herein refers to and encompasses any and all possible combinations of one or more of the associated listed items; and the terms “comprises” and/or “comprising” specify the presence of stated features, but do not preclude the presence or addition of one or more other features.

A “computer system” refers to one or more computers, such as one or more physical computers, virtual computers, and/or computing devices. For example, a computer system may be, or may include, one or more server computers, desktop computers, laptop computers, mobile devices, special-purpose computing devices with a processor, cloud-based computers, cloud-based cluster of computers, virtual machine instances, and/or other computing devices. A computer system may include another computer system, and a computing device may belong to two or more computer systems. Any reference to a “computer system” may mean one or more computers, unless expressly stated otherwise. When a computer system performs an action, the action is performed by one or more computers of the computer system.

A “computing device” may be a computer system, hardware, and/or software stored in, or coupled to, a memory and/or one or more processors on one or more computers. As an alternative or addition, a computing device may comprise specialized circuitry. For example, a computing device may be hardwired or persistently programmed to support a set of instructions to perform the functions discussed herein. A computing device may be a standalone component, work in conjunction with one or more other computing devices, contain one or more other computing devices, and/or belong to one or more other computing devices.

A “component” may be hardware and/or software stored in, or coupled to, a memory and/or one or more processors on one or more computers. As an alternative or addition, a component may comprise specialized circuitry. For example, a component may be hardwired and/or persistently programmed with a set of instructions to perform the functions discussed herein. A component may be a standalone component, work in conjunction with one or more other components, contain one or more other components, and/or belong to one or more other components.

The present disclosure is directed to an intravascularly delivered device and/or devices, systems, and methods for delivering, positioning, deploying and/or releasing an intravascularly delivered device. Throughout this disclosure, many examples are described in the context of an LAA occlusion device. One of skill in the art will understand, however, that the described components, features, and principles may also be utilized in other applications. For example, at least some of the embodiments described herein may be utilized for delivering, positioning, and/or deploying an artificial valve for replacing a pulmonary, aortic, or tricuspid valve. Moreover, it will be understood that at least some of the embodiments described herein may be utilized in conjunction with other intravascularly delivered devices, including occlusion devices, valve repair devices, annuloplasty devices, clip devices, and other intravascularly delivered devices not necessarily configured as an LAA occlusion device. Notwithstanding such alternative applications, preferred embodiments described herein are configured to address challenges particularly associated with delivering, positioning, deploying and/or releasing an LAA occlusion device. The embodiments described below are therefore particularly useful for meeting the additional procedural challenges associated with LAA occlusion through an intravascular approach.

An intravascularly delivered device, such as an LAA occlusion device, includes a plurality of electrodes disposed on a surface of the intravascularly delivered device. A medical navigation system may use the plurality of electrodes to determine a position and/or condition of the intravascularly delivered device during navigation of the vasculature to a target site, such as the LAA. The medical navigation system may use the plurality of electrodes to determine a position and/or condition of the intravascularly delivered device during navigation of the vasculature to a target site, such as the LAA. The medical navigation system may use the plurality of electrodes to evaluate contact between the intravascularly delivered device and tissue at the target site, such as a wall of the LAA. A delivery device may include a second plurality of electrodes disposed on a surface of a distal end of the delivery device. The medical navigation system may use the plurality of electrodes to determine a position and/or condition of the distal end of the delivery device during navigation of the vasculature to a target site.

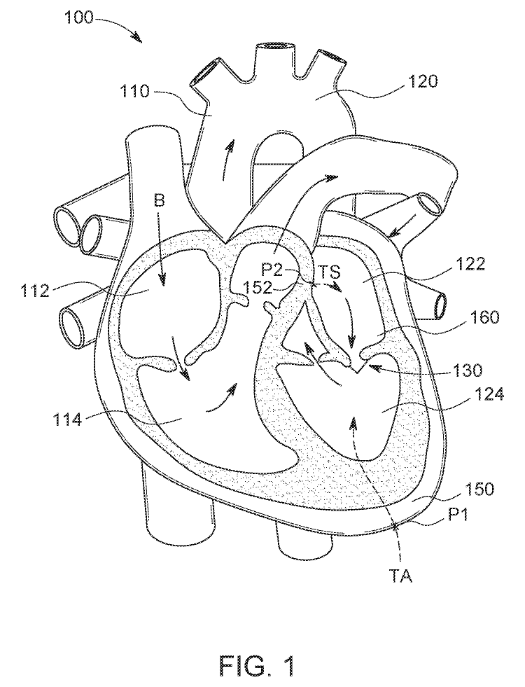

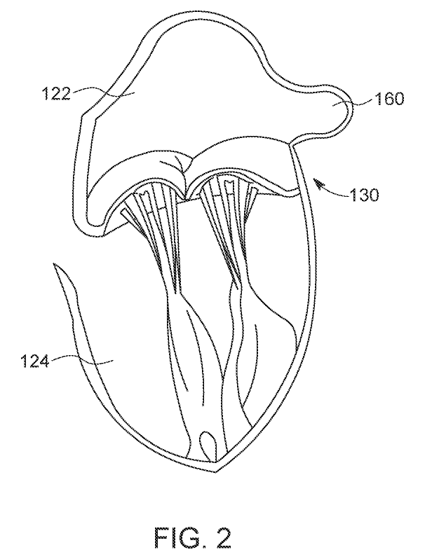

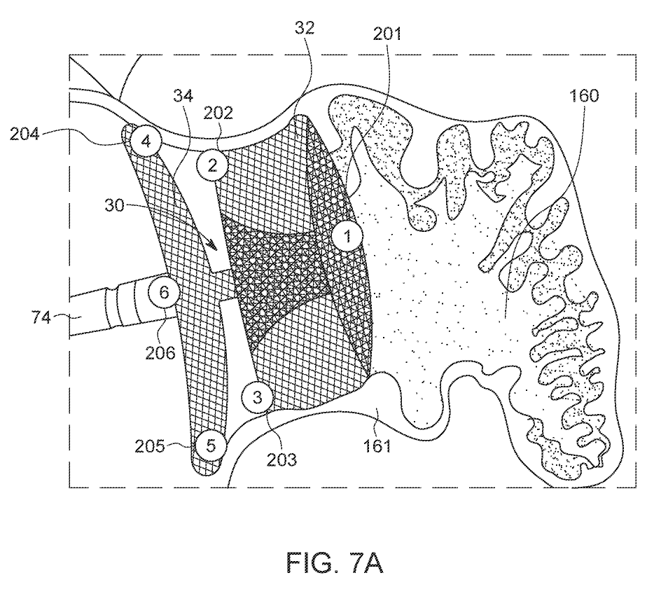

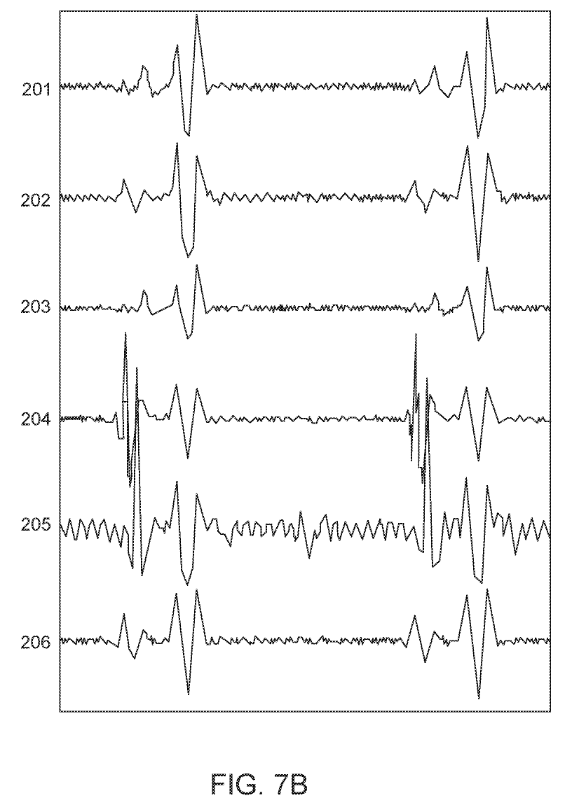

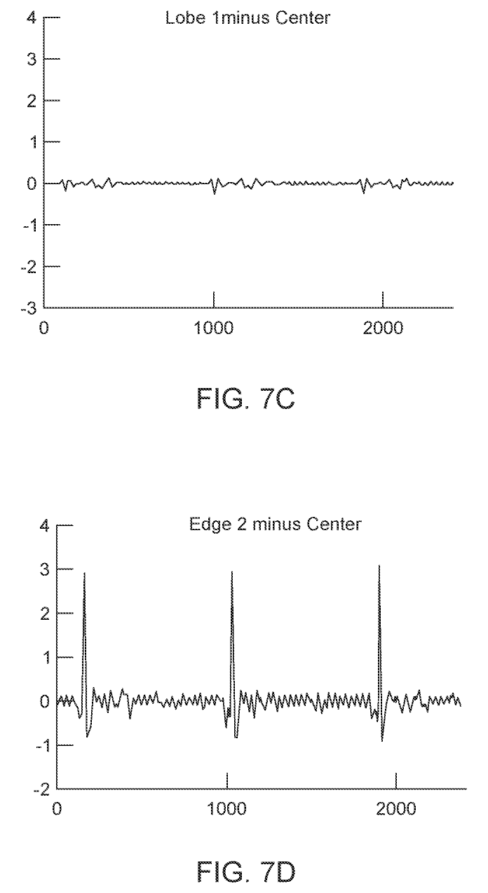

FIG. 1 is a schematic cutaway view of a human heart 100. The human heart includes two atria and two ventricles: the right atrium 112 and the left atrium 122, and the right ventricle 114 and the left ventricle 124. The heart 100 further includes the aorta 110 and the aortic arch 120. The mitral valve 130 is positioned between the left atrium 122 and the left ventricle 124. The mitral valve 130, also known as the bicuspid valve or left atrioventricular valve, is a dual-flap that opens as a result of increased pressure within the left atrium 122 compared to the left ventricle 124. After the left atrium 122 has filled and begins to contract, pressure in the left atrium 122 increases above that in the left ventricle 124, causing the mitral valve 130 to open such that blood passes toward the left ventricle 124. Blood typically flows through the heart 100 in the antegrade direction shown by arrows “B”. Adjacent to the mitral valve 130 is the LAA 160 (best shown in FIGS. 2 and 7A), which empties into the left atrium 122.

A dashed arrow, labeled TA, indicates an example transapical approach for treating or replacing heart tissue. In the transapical delivery of an LAA occlusion device (e.g., LAA occlusion device 30) to the LAA 160, a small incision is made between the ribs and into the apex of the left ventricle 124 at position P1 in the heart wall 150 to deliver the LAA occlusion device 30 to the LAA 160. An alternative path, shown with a second dashed arrow labeled TS, indicates an example transseptal approach with an incision made through the interatrial septum 152 of the heart 100 from the right atrium 112 to the left atrium 122 at position P2. In a transseptal approach, the delivery system may enter the patient through the jugular vein (not shown), proceed through the superior vena cava (shown but not labeled in FIG. 1) and into the right atrium 112, pierce the interatrial septum 152 into the left atrium 122 and approach the LAA 160. More typically, in a transseptal approach, the delivery system may enter the patient through the femoral vein (not shown), proceed through the inferior vena cava (shown but not labeled in FIG. 1) and into the right atrium 112, at which point the procedure is generally the same as described above for the approach from the superior vena cava.

FIG. 2 is a more detailed schematic representation of the left atrium 122 and the left ventricle 124, more closely illustrating the LAA 160. It should be understood that the exact shape and size of the LAA 160 may vary, sometimes significantly, between patients. During normal function, the LAA 160 contracts rhythmically along with the left atrium 122 and blood from the LAA 160 is ejected into the left atrium, then passes through the mitral valve 130 into the left ventricle 124. With each cycle, blood in the LAA 160 is largely or completely emptied out and the mitral valve 130 prevents backflow from the left ventricle 124 to the left atrium 122.

In some patients (e.g., older patients), the right atrium 112 and/or the left atrium 122 of the heart 100 may not beat regularly, a condition known as atrial fibrillation. In some instances, this may result in partial or incomplete ejection of blood from the LAA 160. Stagnant blood in the LAA 160 may form clots, which can ultimately travel to the brain and cause a stroke. To prevent stagnant blood from remaining in and clotting in the LAA 160, an LAA occlusion device can be inserted as a plug in the cavity of the LAA 160.

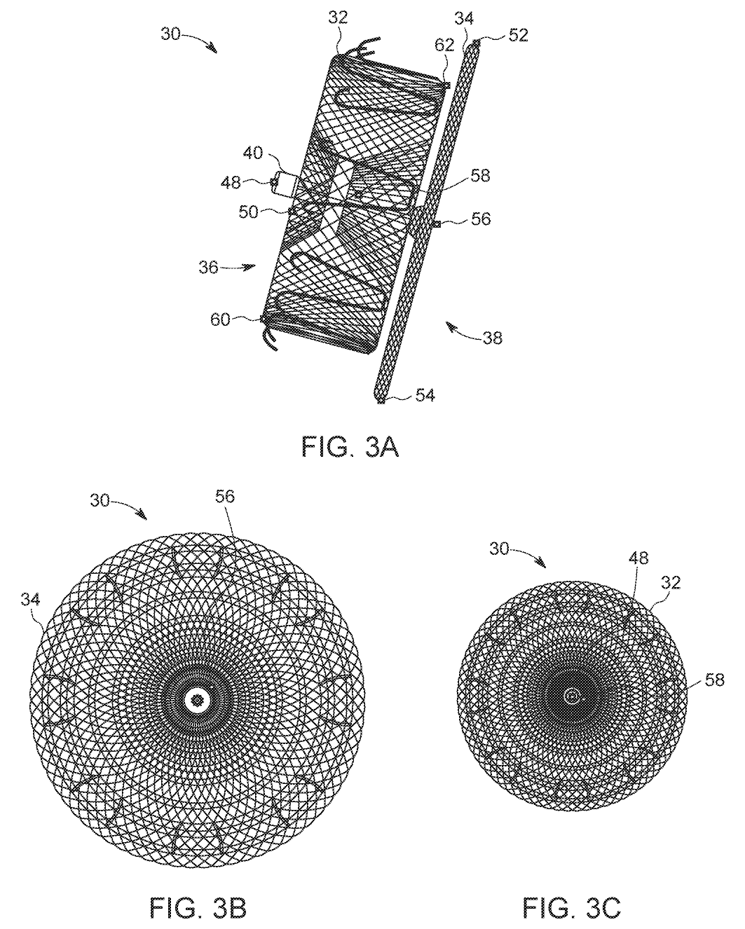

FIG. 3A illustrates an example LAA occlusion device 30. In an LAA occlusion procedure, the LAA occlusion device 30 is deployed in the LAA 160 to reduce the risk of stroke due to atrial fibrillation. The LAA occlusion device 30 may include a disc 34 disposed at a proximal end 38 of the LAA occlusion device 30 and a lobe 32 disposed at a distal end 36 of the LAA occlusion device 30. The lobe 32 is shaped and sized to fit snugly within LAA 160 when fully expanded, and the disc 34 is shaped and sized to cover the opening (or ostium) leading into the LAA 160 when fully expanded. That is, the lobe 32 preferably has an outer diameter in the fully expanded condition that is larger than the interior diameter of LAA 160 such that the lobe 32 is frictionally held in the LAA 160. Similarly, the disc 34 preferably has an outer diameter in the fully expanded condition that is larger than the interior diameter of the ostium of the LAA 160 such that the disc 34 fully covers the opening that leads into the LAA 160. The lobe 32 and the disc 34 may be formed from a mesh including a plurality of strands, wherein at least one strand may be a metal strand. The strands may be braided, interwoven, or otherwise combined to define a generally tubular mesh. While the illustrated embodiment shows the lobe 32 and the disc 34 of the LAA occlusion device 30 in an expanded state, the LAA occlusion device 30 preferably is formed of a shape-memory material (e.g. a nickel-titanium alloy such as nitinol) that enables it to be compressed within a delivery device and to return to its expanded shape when released from the delivery device. A connective element may connect the proximal end of the lobe 32 to the disc 34. The connective element may be configured such that the lobe 32 and the disc 34 are articulable, rotatable, or otherwise movable with respect to the connective element and/or each other. The disc 34 and/or lobe 32 may include one or more fabrics or other materials within the braided mesh, and these fabrics or other materials may help promote tissue ingrowth and/or sealing after implantation of the LAA occlusion device 30. It should be understood that the disclosure provided below may be applicable to a various shapes and styles of LAA occluder devices other than the specific example shown in FIGS. 3A-C. In fact, the disclosure below may apply to any LAA occluder device that is reversibly connected to a delivery device via threading (or via a similar connection mechanism) while the LAA occluder is delivered and deployed into the LAA 160, after which the delivery device is uncoupled from the LAA occluder to allow the delivery device to be withdrawn from the patient while the LAA occluder remains permanently positioned within the LAA 160.

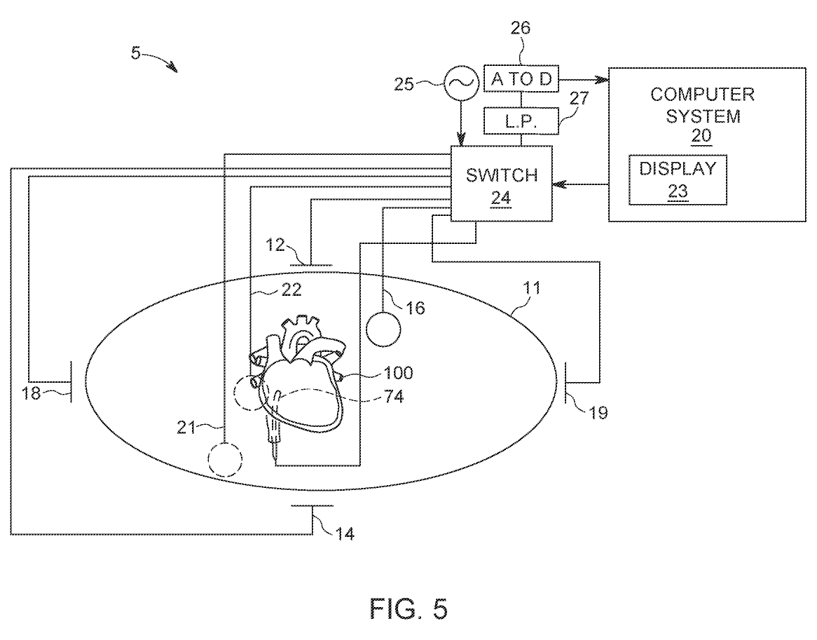

A plurality of electrodes (e.g., electrodes 48, 50, 52, 54, 56, 58, 60, 62) are disposed on a surface of the LAA occlusion device 30. While eight electrodes are illustrated, the plurality of electrodes 48-62 may include any number of electrodes. An electrode is an electrical conductor used to establish electrical contact with and/or carry an electric current into a non-metallic component of a circuit, such as cardiac tissue within the heart 100. A medical navigation system (e.g., medical navigation system 5; FIG. 5) uses the plurality of electrodes 48-62 to determine their position, and hence the position of the LAA occlusion device 30 based on electrode data collected from the plurality of electrodes 48-62. As an addition or alternative, the medical navigation system may use the plurality of electrodes 48-62 to evaluate contact between the LAA occlusion device 30 and cardiac tissue, such as a wall of the LAA 160.

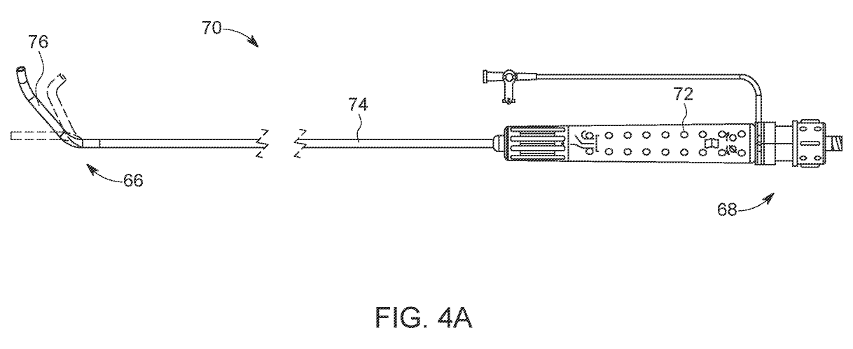

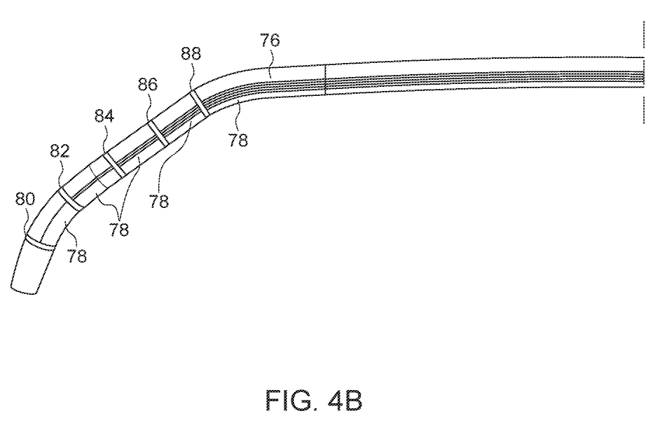

The plurality of electrodes 48-62 are configured to be electrically coupled to a drive source, such as the signal generator 25 of the medical navigation system 5 of FIG. 5. In some embodiments, one or more wires (not shown in FIGS. 3A-C) of the LAA occlusion device 30 electrically couple one or more of the plurality of electrodes 48-62 to one or more wires (not shown in FIGS. 4A-B) of a delivery device (e.g., delivery device 70). The one or more wires of the delivery device may electrically couple the plurality of electrodes 48-62 to the medical navigation system, which is configured to drive the electrodes 48-62 and collect electrode data therefrom. An example medical navigation system is described in greater detail hereinafter.

The plurality of electrodes 48-62 may include one or more electrodes 48-50 disposed on a distal surface of the LAA occlusion device 30. As an alternative or addition, the plurality of electrodes 48-62 may include one or more electrodes 52-54 disposed on an edge surface (e.g., a radially outer edge surface) of the disc 34. As an alternative or addition, the plurality of electrodes 48-62 may include one or more electrodes 56 disposed on a proximal surface of the LAA occlusion device 30. As an alternative or addition, the plurality of electrodes 48-62 may include one or more electrodes 58-62 disposed on a side surface (e.g., a radially outer side surface) of the lobe 32. As an alternative or addition, the plurality of electrodes 48-62 may include one or more other electrodes disposed on another surface of the LAA occlusion device 30. FIG. 3B illustrates the proximal end of the example LAA occlusion device 30, including an electrode 56 disposed on the proximal surface of the LAA occlusion device 30 at or near the radial center of the disc 34. FIG. 3C illustrates the distal end of the example LAA occlusion device 30. Electrode 48 is disposed on the distal tip of the LAA occlusion device 30, such as on the distal screw or clamp 40 of the LAA occlusion device 30. Electrode 50 is disposed adjacent to the base of the distal screw or clamp 40.

The LAA occlusion device 30 may include one or more insulation barriers between one or more of the plurality of electrodes 48-62 and one or more metallic components of the LAA occlusion device 30. As an alternative or addition, one or more conductive elements of the LAA occlusion device 30 may function as one or more of the plurality of electrodes 48-62, such as by electrically coupling the conductive element to a drive source with an isolated electrical wire. For example, one or more stabilizing wires of the LAA occlusion device 30 may be converted into an electrode. It should be understood that, as used herein, the term stabilizing wires may be used synonymously with hooks or anchors that are configured to frictionally engage tissue of the LAA to help anchor the occlusion device 30 in place and resist migration of the occlusion device 30 into the left atrium 122. As is described in greater detail below, one or more electrodes may be used with the occlusion device 30 to assist with, for example, detecting or confirming contact of the occlusion device 30 with tissue of the LAA. Although the electrodes are typically shown and described as separate components, in some embodiments, the stabilizing wires (or hooks or anchors) may form an electrode (or a portion thereof) so that contact between the stabilizing wires and tissue may be detectable via examples of using electrodes described herein. In some embodiments, a distal screw or clamp 40, positioned at or near the radial center of the distal surface of the LAA occlusion device 30, is converted into an electrode 48. As an alternative or addition, an electrode 48 at about the radial center of the distal surface of the LAA occlusion device 30 is positioned at or near the distal screw or clamp 40.

In some examples, the plurality of electrodes 48-62 includes one or more reference electrodes. A reference electrode, also referred to as the ‘indifferent electrode’, has a stable and/or known electrode potential. Data collected from a reference electrode may be used to obtain an accurate measurement using data collected from another electrode typically referred to as an indicator electrode, also referred to as a recording electrode. The usage of a reference electrode for analyzing electrode data is described in greater detail hereinafter.

FIG. 4A illustrates an example delivery device 70. The example delivery device 70 includes a handle 72 at a proximal end 68 of the example delivery device 70. In some embodiments, the example delivery device 70 is configured to deliver an LAA occlusion device 30 to the vicinity of LAA 160 for deployment into the LAA 160. As an addition or alternative, the delivery device 70 may be tailored to deliver any other intravascularly delivered device. The example delivery device 70 includes a catheter 74 that extends between a distal end 66 and the handle 72 of the example delivery device 70, wherein the handle 72 remains outside the patient. In some embodiments, the catheter 74 is a steerable catheter with a flexible, steerable catheter tip 76 at the distal end 66 of the delivery device 70. The catheter 74 has a lumen therethrough that allows the LAA occlusion device 30 to be passed through the delivery device in a compressed configuration.

The lumen of the catheter 74 may further accommodate an inner rod that terminates in a plunger that is used to deploy the LAA occlusion device 30 by translating the LAA occlusion device 30 distally from the catheter 74. As an alternative or addition, the catheter 74 may include a delivery sheath that is retracted to expose and deploy the LAA occlusion device 30. As an alternative or addition, the delivery device 70 may include another structure for translating the LAA occlusion device 30 out from the catheter 74, such as a magnet, a fastener, a blunt tip, or any other suitable mechanism. In some embodiments, a push rod or wire terminates in a threaded tip that is threadedly coupled to a threaded fastener at the radial center of the proximal disc 34. In these embodiments, the push rod may be pushed through the delivery device to push the LAA occlusion device 30 through the delivery device, and the LAA occlusion device 30 may remain threadedly coupled to the push rod until the push rod is rotated to decouple the threaded tip of the push rod from the threaded fastener of the LAA occlusion device 30. The catheter 74 may be formed of any known material for building catheters, including biocompatible polymers and/or metals such as stainless steel. Prior to deployment from the catheter 74, the LAA occlusion device 30 is contained within the lumen of catheter 74 in a compressed configuration. When the distal end 66 of the delivery device 70 is properly positioned relative to the LAA 160, the LAA occlusion device 30 may be urged forward through the catheter 74.

FIG. 4B illustrates a distal portion of the catheter 74 that includes the catheter tip 76. A plurality of electrodes (e.g., electrodes 80, 82, 84, 86, 88) are disposed on a surface of the catheter tip 76. While five electrodes 80-88 are illustrated, the plurality of electrodes 80-88 may include two, three, four, six, or any number of electrodes. In some embodiments, one or more of the plurality of electrodes 80-88 have a ring shape that circumscribes the catheter 74. The plurality of electrodes 80-88 may be spaced evenly over a range of the catheter tip 76, such as about 10mm apart from each other.

A medical navigation system (e.g., medical navigation system 5) uses the plurality of electrodes 80-88 to determine an electrode location in three dimensions for each of the plurality of electrodes 80-88. The medical navigation system can determine the position and/or configuration of the catheter tip 76 in the heart 100 based on the electrode locations. In some embodiments, when the catheter tip 76 is steerable, the specific configuration of the steerable catheter tip 76 may be determined.

In some embodiments, one or more wires 78 of the delivery device 70 electrically couple one or more of the plurality of electrodes 80-88 to a drive source, such as a drive source controlled by a navigation computer system of the medical navigation system. In some embodiments, the one or more wires 78 include a separate wire connected to each of the plurality of electrodes 80-88. The one or more wires of the delivery device 70 may electrically couple the plurality of electrodes 80-88 to the navigation computer system, which is configured to drive the electrodes and collect data therefrom. The lumen of the catheter 74 may house one or more wires running from the plurality of electrodes 80-88 at the distal end of the catheter 74 through the proximal end of the catheter 74, which may also run through at least a portion of the handle 72. As an addition or alternative, the lumen of the catheter 74 may house one or more wires configured to be electrically coupled to the plurality of electrodes 48-62 of the LAA occlusion device 30. Such wires may run from one or more electrical contacts at the distal end 66 of the delivery device 70 through the proximal end of the catheter 74, which may also run through at least a portion of the handle 72. In some embodiments, the wires may run along the outer surface of the catheter 74, and/or through a wall of the catheter, in addition or as an alternative to running through the lumen of the catheter 74. While a lumen of the catheter 74 is described herein, the catheter may comprise one or multiple lumens, any of which may function as described herein. For example, a secondary lumen of the catheter 74 may house the one or more wires as described herein.

FIG. 5 is a schematic diagram of an example medical navigation system. The medical navigation system 5 provides non-fluoroscopic navigation during an intravascular procedure. A patient 11 is schematically depicted as an oval for clarity. When electrical current is applied across two surface electrodes of a pair of electrodes, a voltage gradient is created along the axis between the electrodes. Three sets of surface or patch electrodes are shown as a first pair of electrodes 18, 19 along a Y-axis; a second pair of electrodes 12, 14 along an X-axis; and a third pair of electrodes 16, 22 along a Z-axis. The X-axis, Y-axis, and Z-axis form three orthogonal axes (X-Y-Z). The patient 11 may be positioned such that the patient’s heart 100 is generally near the center between one or more pairs of the electrodes. Patch electrode 16 is disposed on a front surface of the patient 11 that is closest to the reader viewing FIG. 5, and patch electrode 22 is shown in outline form to show its placement on a back surface of the patient 11. The heart 100 of patient 11 lies between these various sets of patch electrodes 18, 19, 12, 14, 16, 22. An additional patch electrode 21, which may be referred to as a “belly” patch, “ground patch”, or “reference patch”, is also illustrated. Each patch electrode 18, 19, 12, 14, 16, 22, 21 is independently connected to a multiplex switch 24. During an intravascular procedure, the patient 11 may have most or all of a conventional surface 12-lead ECG system (not shown) in place, and this ECG information may be available to the navigation computer system 20.

Each patch electrode 18, 19, 12, 14, 16, 22, 21 is coupled to the switch 24, and pairs of electrodes (18, 19), (12, 14), and (16, 22) are selected by software running on the navigation computer system 20, which couples these electrodes 18, 19, 12, 14, 16, 22 to the signal generator 25. A pair of electrodes, for example electrodes 18 and 19, may be excited by the signal generator 25 and they generate a field in the body of the patient 11, including the heart 100. During the delivery of a current pulse, the remaining patch electrodes 12, 14, 16, 22 are referenced to the belly patch electrode 21, and the voltages impressed on these remaining electrodes 12, 14, 16, 22 are measured. A suitable low pass filter 27 or software processes the voltage measurements to remove electronic noise and cardiac motion artifact from the measurement signals. The filtered voltage measurements are transformed to digital data by the analog-to-digital or A-to-D converter 26. As an addition or alternative, other signal processing methods may be employed. In this fashion, the various patch electrodes 18, 19, 12, 14, 16, 22 are divided into driven and non-driven electrode sets. While a pair of electrodes is driven by the signal generator 25, the remaining non-driven electrodes are used as references to synthesize the orthogonal drive axes.

The belly patch electrode 21 is seen in the figure as an alternative to a fixed intra-cardiac electrode. In many instances, a coronary sinus electrode or another fixed electrode in the heart 100 can be used as a reference for measuring voltages and displacements. All of the raw patch voltage data is measured by the A-to-D converter 26 and stored in the navigation computer system 20 under the direction of software. This electrode excitation process occurs rapidly and sequentially as alternate sets of patch electrodes 18, 19, 12, 14, 16, 22 are selected, and the remaining members of the set are used to measure voltages. This collection of voltage measurements may be referred to herein as the “patch data set.” The software has access to each individual voltage measurement made at each individual patch electrode 18, 19, 12, 14, 16, 22 during each excitation of each pair of electrodes 18, 19, 12, 14, 16, 22.

The raw patch data is used to determine the “raw” location in three spaces (X, Y, Z) of the electrodes inside the heart 100, such as the plurality of electrodes 48-62 of the LAA occlusion device 30 and/or the plurality of electrodes 80-88 of the delivery device 70. This process is also referred to as “triangulation.” Triangulation is the process of determining the location of a point by measuring angles from known points. Optical three-dimensional measuring systems use triangulation networks in order to determine spatial dimensions and geometry of objects. Output of at least two of the sensors is considered the point on an object's surface which define a spatial triangle. Within this triangle, the distance between the sensors is the base and is known. By determining the angles between the sensors and the base, the intersection point, and thus the 3D coordinate, is calculated from the triangular relations.

In some embodiments, the navigation computer system 20 controls the signal generator 25 to send an electrical signal through each pair of electrodes (18, 19), (12, 14), and (16, 22) to create a voltage gradient along each of the three axes X, Y and Z, forming a transthoracic electrical field. When the catheter 74 enters the transthoracic electrical field, each catheter electrode 80-88 can sense voltage, timed to the creation of the gradient along each axis. Using electrode data collected from the catheter electrodes 80-88 compared to the voltage gradient on all three axes, the navigation computer system 20 may calculate the three-dimensional position of one or more catheter electrodes 80-88. As an alternative or addition, when the LAA occlusion device 30 enters the transthoracic electrical field, each LAA occlusion device electrode 48-62 can sense voltage, timed to the creation of the gradient along each axis. Using electrode data collected from the LAA occlusion device electrodes 48-62 compared to the voltage gradient on all three axes, the navigation computer system 20 may calculate the three-dimensional position of one or more LAA occlusion device electrodes 48-62. The calculated position for the one or more delivery device electrodes 80-88 and/or LAA occlusion device electrodes 48-62 may be determined simultaneously, and may be performed periodically, such as many times per second.

In some embodiments, the calculated position of one or more delivery device electrodes 80-88 may be used to determine a position and orientation of at least a portion of the delivery device 70, such as but not limited to the catheter tip 76. As an alternative or addition, the calculated position of one or more LAA occlusion device electrodes 48-62 may be used to determine a position and orientation of at least a portion of the LAA occlusion device 30. In some examples, navigation computer system 20 generates an image of the LAA occlusion device 30 and/or the delivery device 70 superimposed on an image of the anatomy of the patient 11. The generated image may be displayed in real-time on a display 23 communicatively coupled with the navigation computer system 20. In some examples, the navigation computer system 20 is provided with a 3D geometry of the anatomy of the patient 11, such as a representation of a portion of the patient’s heart 100, and the generated image includes the LAA occlusion device 30 and/or the delivery device 70 superimposed on a view of the 3D geometry.

As an alternative or addition, the medical navigation system 5 may use a navigation node based on magnetic sensors. As an alternative or addition, one or more electrodes on the LAA occlusion device 30 may be replaced by a plurality of magnetic sensors, such as coils that are configured to be electrically coupled with the medical navigation system 5. A magnetic field is created around the patient 11, such as by using a coil housed below the patient 11. When the magnetic sensors are moved within the magnetic field, electrical current is created and detected by the medical navigation system 5.

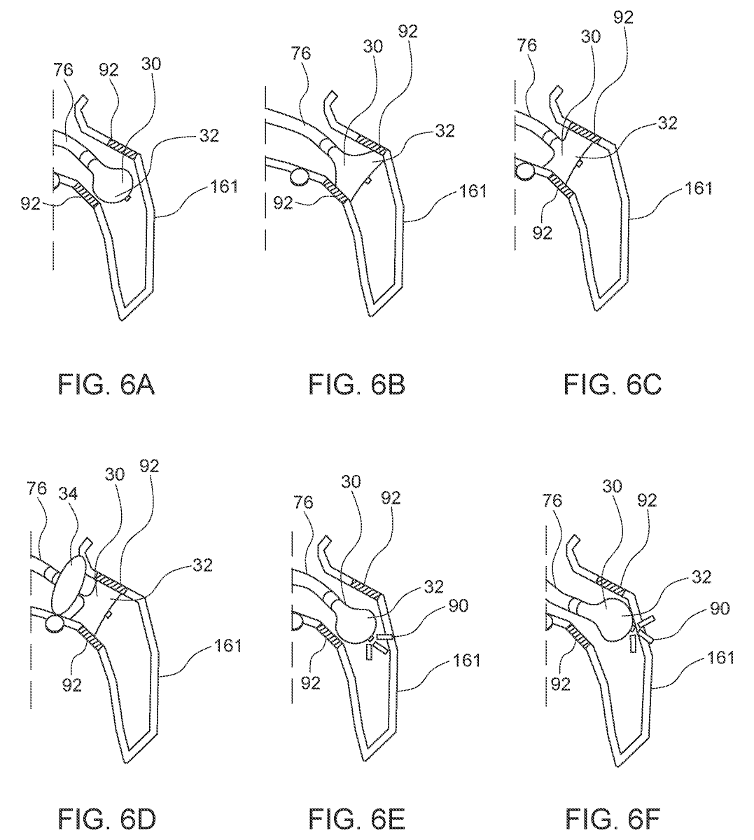

In some examples, the medical navigation system 5 provides guidance for navigating, positioning, and/or deploying an intravascularly delivered device during an intravascular procedure, such as but not limited to an LAA occlusion procedure. FIGS. 6A-6F illustrate stages of an example LAA occlusion device 30 deployment after the catheter tip 76 of the delivery device 70 is positioned in the LAA 160. The delivery device 70 is navigated through the vasculature of the patient using the medical navigation system 5. The navigation may be based on the electrode data corresponding to electrodes 80-88 of the delivery device 70. For example, the catheter tip 76 may be positioned within, adjacent, and preferably coaxial within the LAA based on electrode data displayed by the medical navigation system 5, thus minimizing and/or eliminating the need for fluoroscopy.

In FIG. 6A, the LAA occlusion device 30 is partially translated out of the catheter tip 76 such that the lobe 32 of the LAA occlusion device 30 partially expands into a ball configuration. The ball configuration creates an atraumatic distal tip. The delivery device 70 may be subsequently advanced further in the LAA 160 to a desired deployment position relative to the landing zone 92. For example, the landing zone 92 may be an optimal position for the lobe 32 of the LAA occlusion device 30 to be deployed within the LAA 160. The electrode 48 disposed at the distal tip and the electrode 50 disposed adjacent to the distal screw or clamp 40 create a local dipole signal at the distal end of the LAA occlusion device 30. The navigation computer system 20 processes electrode data from electrodes 48-50 to determine the position of the distal screw 40 or clamp in the LAA 160 while reducing and/or eliminating reliance on fluoroscopy or ultrasound imaging. The medical navigation system 5 may display, on the display 23 of the navigation computer system 20 and/or on one or more other displays, navigation guidance information generated based on the electrode data.

When advancing the LAA occlusion device 30 in the ball configuration, minimizing inadvertent contact with the LAA wall 161 is desired. For example, such inadvertent contact may indicate that the LAA occlusion device 30 is too deep in the LAA 160, or that the LAA occlusion device 30 is exerting unwanted pressure against the distal LAA wall 161. In some embodiments, by using electrode 48 as a reference electrode and electrode 50 as an indicator electrode, the navigation computer system 20 can detect inadvertent contact between the distal screw or clamp 40 and the LAA wall 161. FIGS. 6E-6F illustrates an impedance field 90 that is detectable by the navigation computer system 20 when the navigation computer system 20 controls the driving of corresponding electrodes 48-50. The navigation computer system 20 can detect inadvertent contact, shown in FIG. 6F, by analyzing electrode data describing the impedance field 90. In some examples, the navigation computer system 20 detects local sharp electrogram cardiac signals and modification of the dipole impedance associated with the corresponding electrodes 48-50.

When such contact is detected at this stage, the navigation computer system 20 may notify the physician operating the delivery device 70 of the contact. For example, electrode data collected by the navigation computer system 20 may include intracardiac electrograms recorded by the indicator electrode 48 and the reference electrode 50. The navigation computer system 20 may generate navigation guidance information regarding inadvertent contact between the distal tip of the LAA occlusion device 30 and the LAA wall 161 at the indicator electrode 48 disposed on the distal screw or clamp 40. For example, the navigation computer system 20 may provide real-time feedback regarding inadvertent contact on a display 23 communicatively coupled to the navigation computer system 20.

After reaching the desired deployment position relative to the landing zone 92 with the LAA occlusion device 30 in the ball configuration of FIG. 6A, the physician may further deploy the LAA occlusion device 30 such that the lobe 32 first expands into the generally triangular configuration of FIG. 6B and then fully expands into the generally cylindrical configuration of FIG. 6C. After the lobe 32 deployment is complete, the electrodes 58-62 disposed on the side surface of the lobe 32 are expected to contact the LAA wall 161. In some embodiments, by using the electrodes 58-62 disposed on the side surface of the lobe 32 as indicator electrode/s, the navigation computer system 20 can evaluate contact between the lobe 32 and the LAA wall 161. In some embodiments, electrode 50 is used as a reference electrode for electrodes 58-62. The electrode data collected by the navigation computer system 20 may include intracardiac electrograms recorded by the indicator electrodes 58-62 and the reference electrode 50. The navigation computer system 20 may generate navigation guidance information regarding contact quality between the lobe 32 and the LAA wall 161 at the indicator electrodes 58-62 disposed on the side surface of the lobe 32. For example, the navigation computer system 20 may provide real-time feedback regarding contact quality on a display 23 communicatively coupled to the navigation computer system 20.

In FIG. 6D, the physician deploys the disc 34 of the LAA occlusion device 30. The completion of the disc 34 deployment allows electrodes 52-56 disposed on a surface of the disc 34 to be exposed. Specifically, the one or more electrodes 52-54 disposed on an edge surface of the disc 34 are expected to contact the LAA wall 161. In some embodiments, by using the electrode 56 disposed near the center of the proximal surface of the disc 34 as a reference electrode and one or more electrodes 52-54 disposed on the edge surface of the disc 34 as indicator electrode(s), the navigation computer system 20 can evaluate contact between the disc 34 and the LAA wall 161 at the ostium of the LAA wall 161. When contact is detected at this stage, the navigation computer system 20 may notify the physician operating the delivery device 70 of the contact. The electrode data collected by the medical navigation system 5 may include intracardiac electrograms recorded by the indicator electrodes 52-54 and the reference electrode 50. The navigation computer system 20 may generate navigation guidance information regarding contact quality between the disc 34 and the LAA wall 161 at the indicator electrodes 52-54 disposed on the edge surface of the disc 34. For example, the navigation computer system 20 may provide real-time feedback regarding contact quality on a display 23 communicatively coupled to the navigation computer system 20.

A more detailed explanation of evaluating contact quality is provided with respect to FIGS. 7A-7D. FIG. 7A illustrates an example set of electrodes 201-206 disposed on the surface of an example LAA occlusion device 30. Electrode 201 is a reference electrode disposed adjacent to the base of the distal screw or clamp of the LAA occlusion device 30. Electrodes 202-203 are disposed on a side surface of the lobe 32 of the LAA occlusion device 30. Electrodes 204-205 are disposed on an edge surface of the disc 34 of the LAA occlusion device 30. Electrode 206 is a reference electrode disposed near the center of the proximal surface of the disc 34 of the LAA occlusion device 30.

FIG. 7B illustrates example electrocardiogram data corresponding to the electrodes 201-206. The electrode data collected by the navigation computer system 20 may include intracardiac electrograms recorded by the indicator electrodes 202-205 and the reference electrodes 201, 206. FIG. 7C illustrates a graph showing poor contact quality between the lobe 32 of the LAA occlusion device 30 at electrode 202 and the LAA wall 161. The graph is generated by subtracting the electrical signals for electrode 201 from the electrical signals for electrode 202, where electrode 201 functions as the reference electrode and electrode 202 functions as the indicator electrode. FIG. 7D illustrates a graph showing good contact quality between the disc 34 of the LAA occlusion device 30 at electrode 205 and the LAA wall 161. The amplitude of the periodic spikes indicates a degree of contact. For example, when the LAA occlusion device 30 exerts more pressure at a specific electrode, the corresponding amplitude will be higher. The graph is generated by subtracting the electrical signals for electrode 206 from the electrical signals for electrode 205, where electrode 206 functions as the reference electrode and electrode 205 functions as the indicator electrode. The magnitude of the subtracted electrical signals in FIGS. 7C-7D provides a quantified metric describing a degree of contact between the corresponding electrode 202, 205 and the LAA wall 161. In some embodiments, the navigation computer system 20 generates and displays real-time electrode data and/or navigation guidance information regarding contact quality on a display 23 communicatively coupled to the navigation computer system 20. As used herein, the term electrode data may include raw electrode data and/or processed electrode data collected from any one or any combination of electrodes 48-62 of an LAA occlusion device 30, electrodes 80-88 of a delivery device 70, and/or electrodes 18, 19, 12, 14, 16, 22, 21 of the medical navigation system 5.

In some embodiments, the medical navigation system 5 is used to standardize device placement confirmation techniques, such as but not limited to the traction or tension test (which may also be referred to as the “tug” test). The traction test is a procedure performed by the physician after the disc 34 is fully deployed but before releasing (e.g., unscrewing) the LAA occlusion device 30 from the delivery cable or push rod within the delivery device 70. The physician applies a clinically relevant force, such as by pulling or tugging the delivery cable or push rod (and optionally the delivery device 70), and subjectively evaluates whether the resistance that the physician perceives is sufficient to indicate secure fixation of the LAA occlusion device 30 within the LAA.

The techniques described herein enable quantification of the contact with and/or pressure exerted on the LAA wall 161 by the corresponding electrodes of the LAA occlusion device 30. For example, device placement confirmation techniques may be performed while observing output data presented on the display 23 of the medical navigation system 5. In some examples, one or more standardized values, such as one or more amplitudes of subtracted electrical signals, may be set as a sufficiency threshold. A sufficiency threshold is a value for a parameter that indicates secure fixation of the LAA occlusion device 30. The sufficiency threshold may correspond to changes in electrical signals observed when no pressure is applied to the delivery cable or push rod and/or delivery device 70, allowing the LAA occlusion device 30 to rest as deployed. As an alternative or addition, the sufficiency threshold may correspond to electrical signals observed when a physician performs the tug test. As an alternative or addition, the sufficiency threshold may correspond to electrical signals observed when a standardized amount of pressure is applied to the delivery device 70 in a proximal direction. In some embodiments, one of the indicator electrodes is configured in a bipolar fashion with a reference electrode. A three-dimensional shadow of the bipolar configuration location in the control non-traction or non-tug situation will be created by the navigation computer system 20 before initiation of the traction test. The modification of the impedance signal in combination with the comparison of the electrodes’ new position versus the initial position still indicated by the electrodes shadow will provide an indication of the pulling force generated during the tug test.

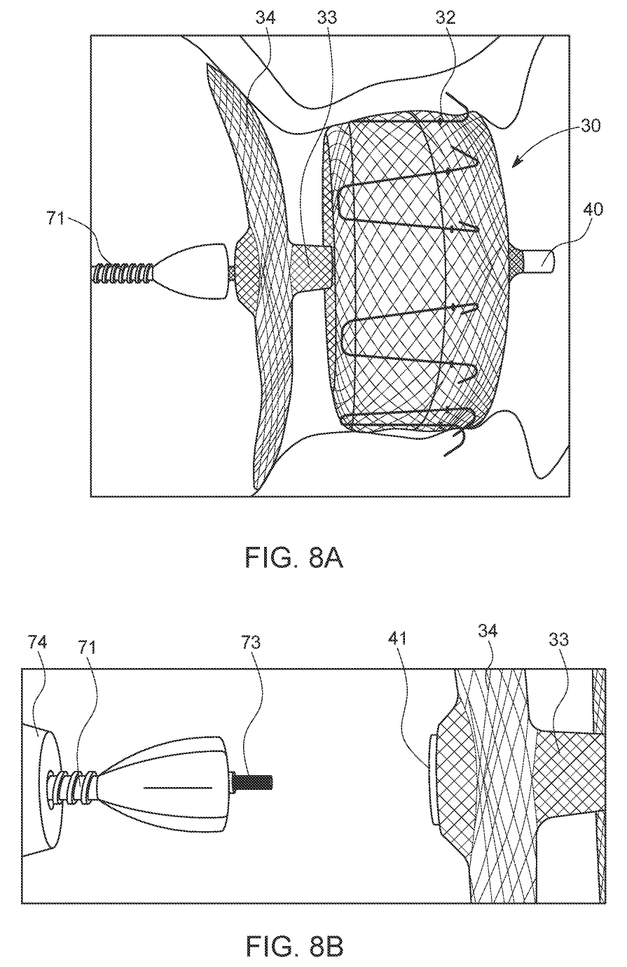

For embodiments in which the LAA occlusion device 30 includes one or more electrodes and/or magnetic sensors on the occlusion device 30 itself (whether or not electrode and/or magnetic sensors are provided on the delivery device 70), the electrodes and/or magnetic sensors on the LAA occlusion device 30 may need to be operably coupled (e.g. via physical wires) to medical navigation system 5 or a component thereof. This may create a difficulty that does not exist for electrodes and/or magnetic sensors on the delivery device 70. For example, as show in FIGS. 8A-8B, a delivery cable 71 which extends through the catheter 74 may terminate in a connector 73, which may be a threaded member or other suitable connector. During delivery of the LAA occlusion device 30, as shown in FIG. 8A, the connector 73 may be coupled to a proximal connector 41 of the disc 34 (which may be a clamp or similar member which includes a complementary connecting feature, such as internal threading, to connector 73). After delivery and deployment of the LAA occluder 30 is completed, including for example after a satisfactory traction test is performed, the connector 73 may be decoupled from the connector 41, for example by rotating the delivery cable 71 to unthread connector 73 from connector 41, at which point the delivery device 70 may be fully removed from the patient. However, when the LAA occlusion device 30 includes electrodes and/or magnetic sensors, one or more conducting wires may need to electrically connect the LAA occlusion device 30 to the navigation system 5 outside the patient, and such wires must also be able to allow for decoupling of the delivery device 70 from the LAA occlusion device 30. Thus, a need may exist to allow for operable coupling (e.g. electrical coupling) of the navigation system 5 to the LAA occlusion device 30 while the LAA occlusion device 30 is physically coupled to the delivery device 70, without impeding the ability for the delivery device 70 to disconnect from the LAA occlusion device 30.

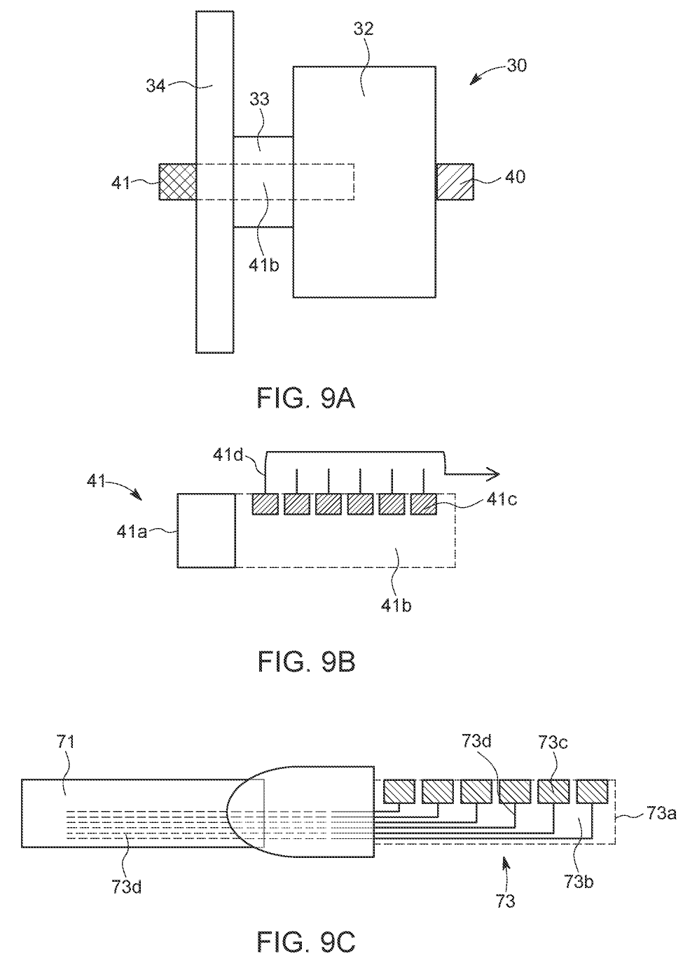

FIGS. 9A-9C illustrate an exemplary mechanism by which the LAA occluder 30 may be electrically coupled to the navigation system 5 via delivery device 70 during delivery, without such connection impeding the release of the LAA occluder 30 from the delivery device 70. For example, a schematic diagram of LAA occluder 30 is shown in FIG. 9A in which the position of connector 41 is not just limited to the disc 34, but in which the connector 41 has an extended length that extends through at least part of the interior of the waist 33 that connects disc 34 to lobe 32, and potentially through at least part of the interior of the lobe 32. As shown in FIGS. 9A and 9B, the proximal connector 41 may include proximal end 41a that is configured to receive the connector 73. In some examples, the proximal end 41a is an opening that leads to an interior, internally threaded, substantially cylindrical tube 41b. Referring in particular to FIG. 9B, one or more electrodes 41c or other conduction members may be positioned on an interior wall of the tube 41b. In some examples, each electrode 41c may be positioned a spaced distance from an adjacent electrode 41c (for example in a linear array), and the number of electrodes 41c may correspond to the number of electrodes on the LAA occluder 30. In the particular example of FIG. 9B, six electrodes 41c are provided, which may correspond, for example, to the six electrodes 201-206 shown in FIG. 7A. Each electrode 41c of the connector 41 may be coupled, for example by a conductive wire 41d, to a corresponding one of the electrodes (e.g. electrodes 201-206) on the LAA occluder 30. Although only one wire 41d is represented in FIG. 9B, it should be understood that each electrode 41c may have such a wire 41d to couple to the corresponding electrode on the LAA occluder 30.

FIG. 9C shows a schematic illustration of a distal end of the delivery system 70, in particular a distal end of the delivery cable 71 including connector 73. As noted above, connector 73 may be an externally threaded shaft, but other mechanisms may be suitable to allow for reversible coupling of the connector 73 to the connector 41. In the illustrated example, an interior channel or lumen 73b may extend through both the delivery cable 71 and at least partially through the connector 73, although the lumen 73b needs to extend to the terminal distal end 73a of the connector 73. The channel or lumen 73b may be configured to allow for one or more wires, such as conducting wires 73d, to extend through the delivery device 70 to physically couple the navigation system 5, or a component thereof, to corresponding electrodes 73c. One or more of the electrodes 73c (or other conduction members) may be positioned at least on an exterior face of the connector 73. As with electrodes 41c, each electrode 73c may be positioned a spaced distance from an adjacent electrode 73c (for example in a linear array), and the number and position of electrodes 73c may generally match the number and position of electrodes 41c. It should be understood that other numbers and configurations (e.g. helical vs. linear array) of electrode positioning may be suitable, and the particular number and positioning of the electrodes shown in FIGS. 9A-9C should not be considered limiting.

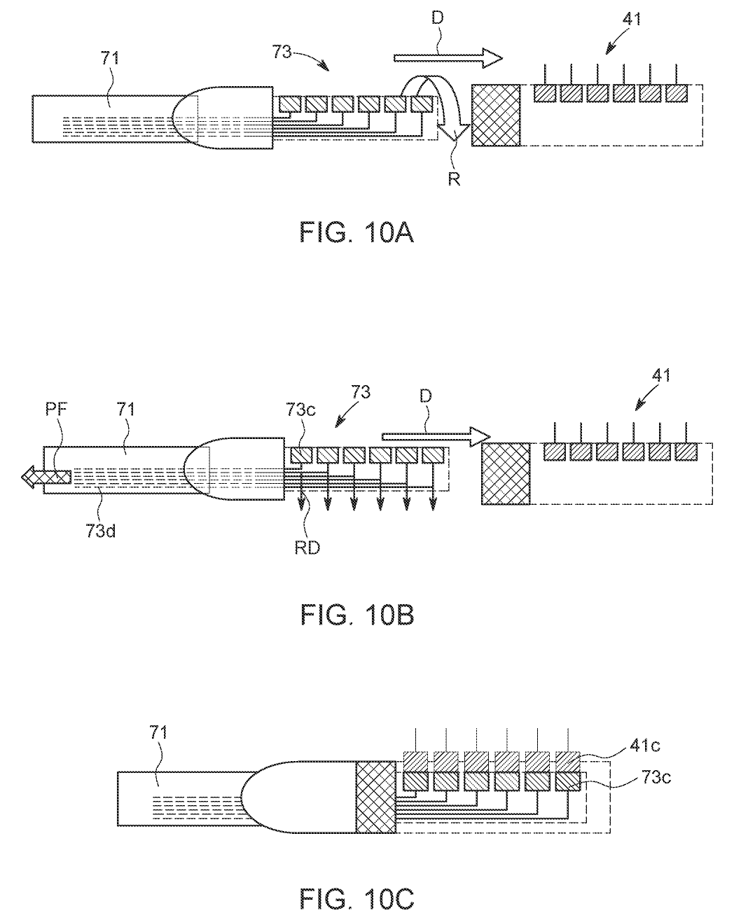

Any suitable mechanism may be used to allow for the connector 73 to reversibly couple to the connector 41 to provide contact between corresponding electrodes 41c, 73c. For example, as mentioned above and shown in FIG. 10A, connector 73 may be an externally threaded male screw-type mechanism and connector 41 may be an internally threaded female screw-type mechanism, such that rotating the connector 73 in a rotational direction R will tend to thread the connector 73 into connector 41 and cause the connector 73 to advance distally D relative to the connector 41. If a screw-type mechanism is used, the threading may be configured so that, when the threading is complete, the electrodes 41c each confront and/or contact a respective electrode 73c (for example as shown in FIG. 10C). It should be understood that the connector 73 may be decoupled from the connector 41 by rotating the connector 73 in the rotational direction opposite to rotational direction R to unthread the connector 73 from the connector 41. Instead of having connector 73 reversibly thread into or out of connector 41, the connector 73 may have a snap fit type of connection in which the connector 73 is pushed distally into the connector 41 (for example without needing any rotation or threading), and one or more tabs or similar features on the connector 73 engage one or more recesses or similar features on connector 41 to engage the connector 73 to the connector 41. In such a push-to-connect configuration, the delivery system 70 may include a retraction mechanism (e.g. on a handle of the delivery system 70), whereby actuating the retraction mechanism temporarily withdraws electrodes 73c away from the outer surface of the connector 73. For example, referring to FIG. 10B, actuating the retraction mechanism may apply a slight proximal force PF on the wires 73d, which in turn may draw the electrodes 73c in a retraction direction RD toward the interior of the connector 73 while the proximal force PF is applied. With this configuration, the connector 73 may be inserted (e.g. by pushing in the distal direction D) into the connector 41 (which may be an unthreaded tube in this example) while the proximal force PF is maintained and the electrodes 73 are in a retracted state so that the electrodes 73 do not hinder the entry of the connector 73 into the connector 41 (and/or the electrodes 73 are not subjected to damage, for example by avoiding scraping along the inner surface of the connector 41 during insertion). Once the connector 73 is fully pushed into the connector 41, which may be confirmed with a tactile or audible click or snap in some examples, the proximal force PF may be released, allowing the electrodes 73c to re-emerge from their retracted position (e.g. opposite the retraction direction RD) into contact with corresponding electrodes 41c, as shown in FIG. 10C. It should be understood that the connection may be reversed by again applying the proximal force PF and pulling the connector 73 proximally from the connector 41. In some examples, the actuator that applies the proximal force PF may have a lock so that the user does not need to maintain force on the actuator to maintain the proximal force PF, which may be particularly useful when the LAA occluder 30 has been fully deployed into the LAA and it is time to disconnect the delivery system 70 from the LAA occluder 30.

The screw-to-connect (or unscrew to disconnect) and the push-to-connect (or pull-to-disconnect) mechanisms described above are just two examples of how the two connectors 73, 41 may be reversibly coupled so that, when coupled, the electrodes 73c of the connector 73 align with and contact the corresponding electrodes 41c of the connector 41, and it should be understood that various other options may be suitable. In some examples, the connector 41 may be an externally threaded male screw-type device and the connector 73 may be an internally threaded screw-type device, although it may not be optimal to leave external threads of a connector exposed to blood flow within the body. Still other connection mechanisms may be suitable. For example, in another configuration, the electrodes 41c, 73c may be pre-connected to their corresponding receptacles and held in place with a cylindrical mandrel ensuring a firm connection between electrodes 41c and electrodes 73c. After the LAA occluder 30 is implanted and the delivery system 70 is ready for disconnection, an unscrewing action of connector 73 may pull out the cylindrical mandrel and thus release the connected electrodes 41c, 73c that are mechanically held in place with the mandrel. This mandrel-type mechanism can be applicable in axial, radial or helical arrangement of the electrodes 41c, 73c. For such a configuration, sufficient clearance between the mandrel and cables may be important to help ensure free rotation of the mandrel.

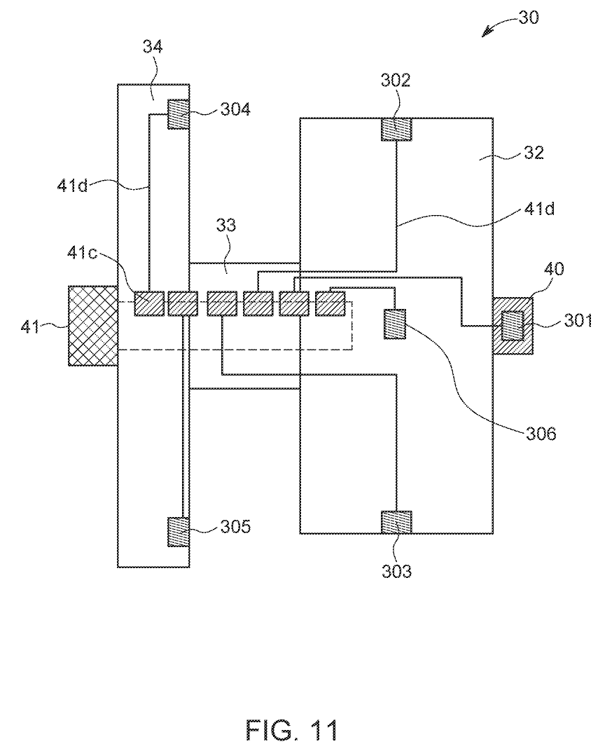

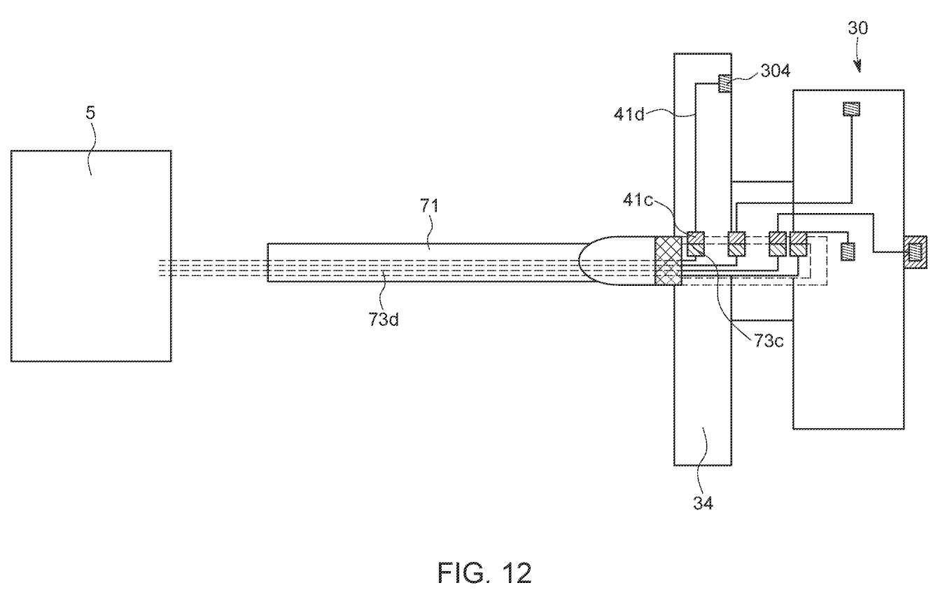

Regardless of the particular mechanism by which the connector 73 is reversibly coupled to the connector 41, once the coupling is complete, as shown in FIG. 10C, each electrode 73c is in contact with a corresponding electrode 41c. Before describing the full connection of the delivery system 70 to the LAA occluder 30, one example of further details of the LAA occluder 30 is provided in connection with FIG. 11, which shows a schematic view of LAA occluder 30. As shown in FIG. 11, the LAA occluder 30 may include a lobe 32, a disc 34, and a waist 33 connecting the two. A distal clamp or fastener 40 may be positioned at a distal end portion of the lobe 32, and may include an electrode 301 (e.g. similar or identical to electrode 201 of FIG. 7A. The lobe 32 may include additional surface electrodes 302, 303, 306 (e.g. similar or identical to electrodes 202, 203 of FIG. 7A). The disc 34 may include additional surface electrodes 304, 305 (e.g. similar or identical to electrodes 204, 205 of FIG. 7A). Electrodes 304, 305 may be configured to contact the tissue surrounding the ostium of the LAA to help confirm contact. Although not shown in FIG. 11, an electrode similar to electrode 206 (which may be a reference or non-tissue-contacting electrode) may also be provided in the configuration of the LAA occluder 30 shown in FIG. 11. Further, in some examples, any of the electrodes 301-306 may function as a reference electrode. It should be understood that the particular configuration of the number and placement of electrodes 301-306 are merely exemplary in FIG. 11. As shown in FIG. 11, each electrode 301-306 on the LAA occluder 30 is coupled, via a respective wire 41d (only two of which are labeled in FIG. 11) to a respective electrode 41c (only one of which is labeled in FIG. 11) of connector 41.

With this configuration, as shown in FIG. 12, when the connector 73 is received within and coupled to connector 41, the electrodes (e.g. electrodes 301-306) of the LAA occluder 30 are electrically coupled to the navigation system 5. It should be understood that, in FIG. 12, only some of the electrodes 301-306 of FIG. 11 are shown for purposes of simplicity. Electrode 304, which may be on the distal surface of the disc 34 of the LAA occluder 30, is described below, but it should be understood that the connection of electrode 304 to the navigation system 5 may be representative of all other electrodes of the LAA occluder 30. As shown in FIG. 12, upon connection of connectors 41, 73, contact between electrode 41c, 73c results in electrical connection between electrode 304 and navigation system 5, first via the wire 41d connecting electrode 304 and electrode 41c, and then via the wire 73d connecting electrode 73c and navigation system 5. Thus, during delivery of the LAA occluder 30 while the LAA occluder 30 is coupled to the delivery system 70, all of the electrodes (e.g. electrodes 301-306) may have an active electrical connection with the navigation system 5 in order to assist with the delivery and deployment of LAA occluder 30, for example as generally described in connection with FIGS. 1-7D. However, once the LAA occluder 30 is suitably deployed into the LAA, the delivery system 70 may be readily decoupled from the LAA occluder 30 to leave the LAA occluder 30 in its final implanted position, without the wired connection between the LAA occluder 30 and the navigation system 5 impeding the ability to decouple the LAA occluder 30 from the delivery system 70. It should be understood that the schematic view of FIG. 12 is not to scale.

Referring briefly again to FIG. 11, as described above, each electrode 41c coupled to connector 41 of the occluder 30 is coupled, via a conducting wire 41d, to a respective one of the electrodes 301-306 of the occluder 30. In some examples, the conducting wires 41d may generally extend through interior spaces of the occluder 30 (e.g. generally within open spaces interior of the braided mesh that forms the main structure of the occluder 30). However, in other examples, some or all of the conducting wires 41d may be braided or otherwise integrated with the other braided wires that form the mesh of the occluder 30.

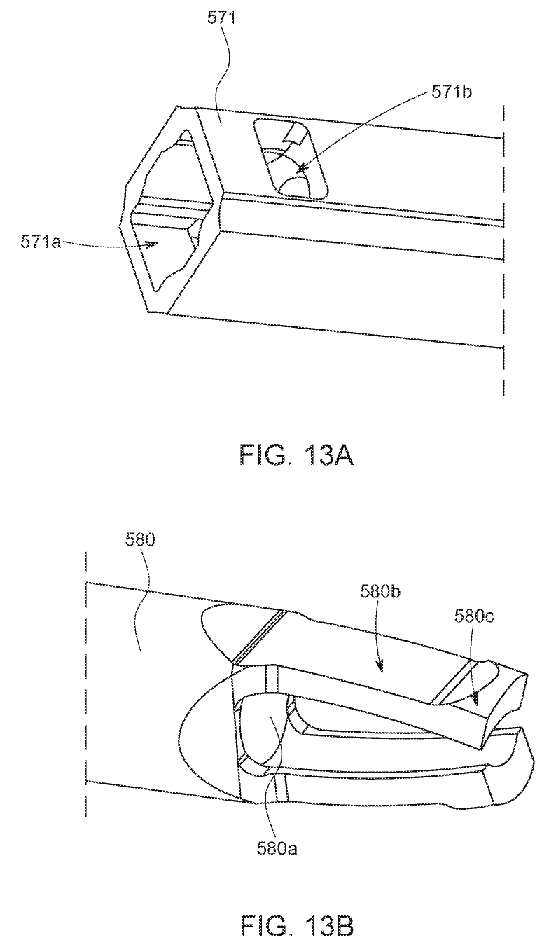

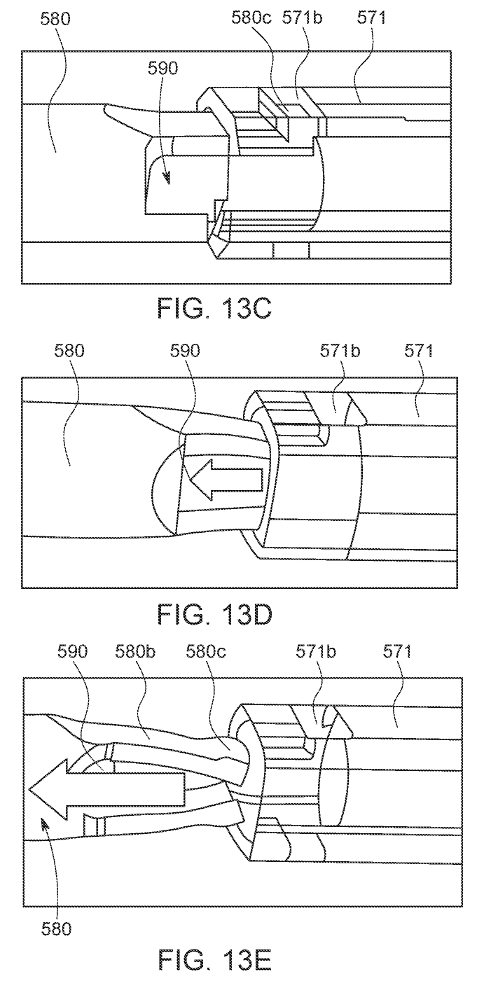

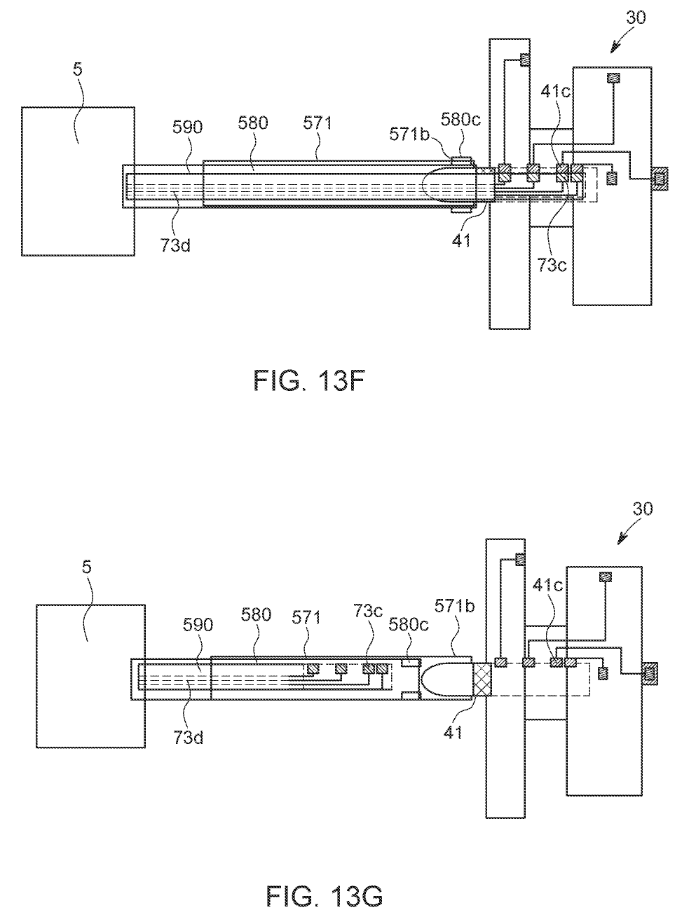

Although various mechanisms for reversibly coupling conducting wires 73d and electrodes 73c to electrodes 41c of LAA occluder 30 for delivery of the LAA occluder 30 are described above, it should be understood that still other mechanisms for reversible coupling may be suitable. FIG. 13A illustrates a structure which may be formed at or near a distal end of a delivery cable 571, which may be otherwise similar or identical to delivery cable 71 described above. FIG. 13A is a cutaway view with the proximal portion of delivery cable 571 omitted, so that a hollow channel 571a is visible, with the hollow channel opening to two tab windows 571b on opposite sides of the delivery cable 571. It should be understood that a threaded connector (which may be generally similar to connector 73) may be positioned at the distal end of the delivery cable 571 (to the right in the view of FIG. 13A), although such a connector is omitted from the figure. And although delivery cable 571 is shown as having a profile of a square or rectangle with rounded edges, in other examples it may be substantially cylindrical.

FIG. 13B illustrates a distal end of a hollow connector shaft 580, which may be a component that does not strictly correspond to components of other embodiments described herein. Hollow connector shaft 580 may be sized to be received within the delivery cable 571 so that the hollow connector shaft 580 can slide distally and proximally relative to the delivery cable 571 while inside the delivery cable 571. Hollow connector shaft 580 may include a central lumen 580a through an entire length thereof, with the distal end of the hollow connector shaft 580 terminating in two flexure arms 580b. The flexure arms 580b may be spring loaded or shape-set to flex inwardly toward each other, such as the configuration shown in FIG. 13B. Each flexure arm 580b may terminate in an outward bump, protrusion, or tab 580c that is shaped to be received within a corresponding tab window 571b of the delivery cable 571. In this relaxed condition, the flexure arms 580b are positioned so that the tabs 580c are positioned inward of the outer diameter of the remainder of the hollow connector shaft 580.

FIGS. 13C-E illustrate three stage of disconnecting the hollow connector shaft 580 from the delivery cable 571. Referring to FIG. 13C, a wire cable 590 is shown extending through the central lumen 580a of the hollow connector shaft and through the hollow channel 571a of the delivery cable 571, while the tabs 580c of the flexure arms 580b are aligned with the tab windows 571b. The wire cable 590 is sized so that it forces the flexure arms 580b outwardly, and the tabs 580c are received within the tab windows 571b, securely locking the hollow connector shaft 580 to the delivery cable 571 as long as the wire cable 590 extends distally beyond the flexure arms 580b. The wire cable 590 may include conducting wires, which may be similar or identical to conducting wires 73d, which may terminate at electrodes, which may be similar or electrodes 73c, and a distal end of the wire cable 590, as described in greater detail in connection with FIGS. 13F-G.

Referring again to FIG. 13C, and as noted above, as long as wire cable 590 extends beyond the distal end of the flexure arms 580b while the tabs 580c are aligned with (and received in) the tab windows 571b, the hollow connector shaft 580 remains fixed to the delivery cable 571. The wire cable 590 may be sized to fit relatively tightly within the lumen 580a of the hollow connector shaft 580, such that the wire cable 590 also remains secure in its position until and unless an intentional pulling force is placed on the wire cable 590 to pull the wire cable 590 proximally. Referring briefly to FIG. 13F, the wire cable 590 (and/or the conducting wires 73d thereof) may extend on one end to navigation system 5, and electrodes 73c may be positioned at or near a distal end of wire cable 590, generally similar to as described in connection with other embodiments herein. As shown in FIG. 13F, the electrodes 73c may be coupled to (and/or in contact with) electrodes 41c in an initial condition of the LAA occluder 30 pre-implantation. In this condition, the delivery cable 571 may be coupled (e.g. threadedly coupled) to connector 41, with the wire cable 590 forcing the tabs 580c of hollow connector shaft 580 into the corresponding tab windows 571b of delivery cable 571. Thus, the configuration of FIG. 13F may correspond to that shown in FIG. 13C, with the hollow connector shaft 580 locked to the delivery cable 571, the electrodes 73c on wire cable 590 electrically connected to the electrodes 41c, and the wire cable 590 held secure in place, for example via friction with the hollow connector shaft 580 (and/or the flexure arms 580b). The LAA occluder 30 may be delivered in this condition, with the various electrodes on the LAA occluder electrically coupled to the navigation system 5 in substantially the same fashion as described in connection with other embodiments herein.

After LAA occluder 30 has been delivered and the LAA occluder 30 needs to be disconnected from the delivery system, the wire cable 590 may first be withdrawn. For example, referring to FIG. 13D, the wire cable 590 may be pulled proximally, which may overcome any friction force applied by the hollow connector shaft 580, allowing the wire cable 590 to withdraw from the LAA occluder 30 and retract proximally within the hollow connector shaft 580 to a position proximal to the flexure arms 580b. Due to the inward bias of the flexure arms 580b, once the wire cable 590 no longer prevents the flexure arms 580b from taking their preferred (e.g. heat-set) shape, the flexure arms 580b will naturally flex inwardly, with the tabs 580c moving inwardly while exiting the tab windows 571b. In this configuration, as shown in FIG. 13D, the hollow connector shaft 580 is no longer fixed to the delivery cable 571. In this condition, as shown in FIG. 13E, the hollow connector shaft 580 and wire cable 590 may be further withdrawn through the delivery cable 571. As noted above, the portion of delivery cable 571 proximal to the distal end is omitted from FIGS. 13C-13E to provide clearer illustration of the hollow connector shaft 580 and the wire cable 590.

FIG. 13G shows the delivery cable 571 still connected to connecter 41, but the wire cable 590 and the hollow connector shaft 580 having been retracted a distance proximally through the interior lumen of the delivery cable 571, generally similar to the condition shown in FIG. 13E. In this condition, although the delivery cable 571 is still mechanically linked to the LAA occluder 30, the navigation system 5 is no longer electrically connected to the LAA occluder 30 because the electrodes 73c, which are positioned on the wire cable 590, have been withdrawn proximally out of contact with their corresponding electrodes 41c on the connector 41 of the LAA occluder 30. After the electrical disconnection shown in FIG. 13G is performed, the delivery cable 571 may be disconnected from the connector 41 of the LAA occluder 30 and withdrawn from the patient, either before, after, or simultaneous with the withdrawal of the hollow connector shaft 580 and/or the wire cable 590 from the patient. It should be understood that this is just one more exemplary mechanism to provide a reversible electrical and mechanical connection between a navigation system and delivery system to an LAA occluder for delivery and deployment, with straightforward mechanical and electrical disconnection of the components after the LAA occluder is suitably deployed within the LAA of the patient.

The disclosure above is generally directed to mechanisms by which an implantable device may be temporarily coupled to a delivery device, and while the components are connected, an uninterrupted electrical or conductive connection may be formed between (i) electrodes (and/or magnetic sensor) that are permanently coupled to the implantable device and (ii) a navigation system outside the patient, via the delivery device. The disclosure above is also generally directed to mechanisms by which the implantable device may be disconnected from the delivery device to leave the implantable device permanently implanted within the patient, without having the features that allow the electrical or conductive connection impeding the ability to achieve that disconnection. Although the disclosure is provided in the context of a delivery device that reversibly couples to an LAA occluder 30, it should be understood that the same features, with or without modification, may be applied to other implantable devices. For example, various other types of closure devices, such as patent ductus arteriosus (“PDA”) occluders, atrial septal defect (“ASD”) occluders, ventricular septal defect (“VSD”) occluders, and patent foramen ovale (“PFO”) occluders, may all be provided with similar or identical features as described above in connection with LAA occluder 30, with or without additional modifications. Still further, other non-occluder implants, such as collapsible and expandable prosthetic heart valves (e.g. prosthetic aortic, pulmonary, mitral, or tricuspid valves), and valve repair devices (e.g. transcatheter edge-to-edge repair devices such as leaflet clips or leaflet fixation device) may also be provided with features similar to those described above, with or without modification, to provide for a conductive connection between electrodes (or magnetic sensors) on the implant and an external navigation system while the delivery device is coupled to the implantable device, without such connection hampering the disconnection when ready to disconnect the implantable device from the delivery device for final implantation.

The techniques described herein may be implemented by one or more special-purpose computing devices. The special-purpose computing devices may be hard-wired to perform one or more techniques described herein, including combinations thereof. Alternatively and/or in addition, the one or more special-purpose computing devices may include digital electronic devices such as one or more application-specific integrated circuits (ASICs) or field-programmable gate arrays (FPGAs) that are persistently programmed to perform the techniques. Alternatively and/or in addition, the one or more special-purpose computing devices may include one or more general-purpose hardware processors programmed to perform the techniques described herein pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices, and/or any other device that incorporates hard-wired or program logic to implement the techniques.

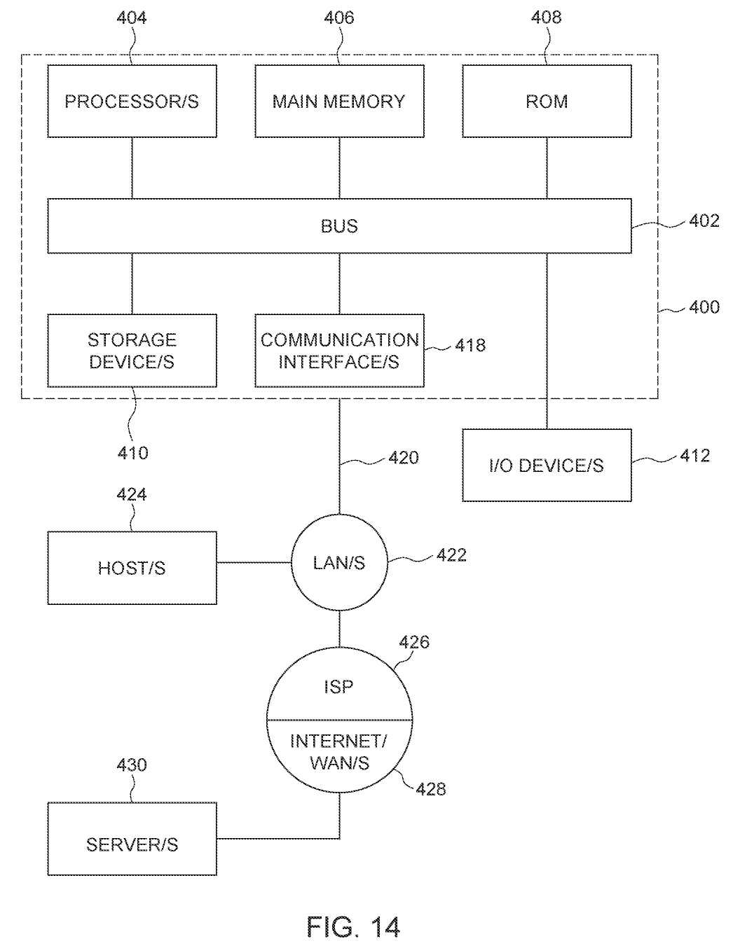

FIG. 14 is a block diagram that illustrates a computer system upon which one or more examples may be implemented. The computer system 400 includes a bus 402 or other communication mechanism for communicating information, and one or more hardware processors 404 coupled with bus 402 for processing information, such as computer instructions and data. The processor/s 404 may include one or more general-purpose microprocessors, graphical processing units (GPUs), coprocessors, central processing units (CPUs), and/or other hardware processing units.

The computer system 400 also includes one or more units of main memory 406 coupled to the bus 402, such as random-access memory (RAM) or other dynamic storage, for storing information and instructions to be executed by the processor/s 404. Main memory 406 may also be used for storing temporary variables or other intermediate information during execution of instructions to be executed by the processor/s 404. Such instructions, when stored in non-transitory storage media accessible to the processor/s 404, turn the computer system 400 into a special-purpose machine that is customized to perform the operations specified in the instructions. In some embodiments, main memory 406 may include dynamic random-access memory (DRAM) (including but not limited to double data rate synchronous dynamic random-access memory (DDR SDRAM), thyristor random-access memory (T-RAM), zero-capacitor (Z-RAM™)) and/or non-volatile random-access memory (NVRAM).

The computer system 400 may further include one or more units of read-only memory (ROM) 408 or other static storage coupled to the bus 402 for storing information and instructions for the processor/s 404 that are either always static or static in normal operation but reprogrammable. For example, the ROM 408 may store firmware for the computer system 400. The ROM 408 may include mask ROM (MROM) or other hard-wired ROM storing purely static information, programmable read-only memory (PROM), erasable programmable read-only memory (EPROM), electrically-erasable programmable read-only memory (EEPROM), another hardware memory chip or cartridge, or any other read-only memory unit.

One or more storage devices 410, such as a magnetic disk or optical disk, is provided and coupled to the bus 402 for storing information and/or instructions. The storage device/s 410 may include non-volatile storage media such as, for example, read-only memory, optical disks (such as but not limited to compact discs (CDs), digital video discs (DVDs), Blu-ray discs (BDs)), magnetic disks, other magnetic media such as floppy disks and magnetic tape, solid-state drives, flash memory, optical disks, one or more forms of non-volatile random-access memory (NVRAM), and/or other non-volatile storage media.

The computer system 400 may be coupled via the bus 402 to one or more input/output (I/O) devices 412. For example, the I/O device/s 412 may include one or more displays for displaying information to a computer user, such as a cathode ray tube (CRT) display, a Liquid Crystal Display (LCD) display, a Light-Emitting Diode (LED) display, a projector, and/or any other type of display.

The I/O device/s 412 may also include one or more input devices, such as an alphanumeric keyboard and/or any other keypad device. The one or more input devices may also include one or more cursor control devices, such as a mouse, a trackball, a touch input device, or cursor direction keys for communicating direction information and command selections to the processor 404 and for controlling cursor movement on another I/O device (e.g. a display). A cursor control device typically has degrees of freedom in two or more axes, (e.g. a first axis x, a second axis y, and optionally one or more additional axes z), that allows the device to specify positions in a plane. In some embodiments, the one or more I/O device/s 412 may include a device with combined I/O functionality, such as a touch-enabled display.

Other I/O device/s 412 may include a fingerprint reader, a scanner, an infrared (IR) device, an imaging device such as a camera or video recording device, a microphone, a speaker, an ambient light sensor, a pressure sensor, an accelerometer, a gyroscope, a magnetometer, another motion sensor, or any other device that can communicate signals, commands, and/or other information with the processor/s 404 over the bus 402.

The computer system 400 may implement the techniques described herein using customized hard-wired logic, one or more ASICs or FPGAs, firmware, and/or program logic that causes the computer system 400 to be a special-purpose machine. In some examples, the techniques herein are performed by the computer system 400 in response to the processor/s 404 executing one or more sequences of one or more instructions contained in main memory 406. Such instructions may be read into main memory 406 from another storage medium, such as the one or more storage device/s 410. Execution of the sequences of instructions contained in main memory 406 causes the processor/s 404 to perform the process steps described herein. In alternative embodiments, hard-wired circuitry may be used in place of or in combination with software instructions.

The computer system 400 also includes one or more communication interfaces 418 coupled to the bus 402. The communication interface/s 418 provide two-way data communication over one or more physical or wireless network links 420 that are connected to a local network 422 and/or a wide area network (WAN), such as the Internet. For example, the communication interface/s 418 may include an integrated services digital network (ISDN) card, cable modem, satellite modem, or a modem to provide a data communication connection to a corresponding type of telephone line. Alternatively and/or in addition, the communication interface/s 418 may include one or more of: a local area network (LAN) device that provides a data communication connection to a compatible local network 422; a wireless local area network (WLAN) device that sends and receives wireless signals (such as electrical signals, electromagnetic signals, optical signals or other wireless signals representing various types of information) to a compatible LAN; a wireless wide area network (WWAN) device that sends and receives such signals over a cellular network; and other networking devices that establish a communication channel between the computer system 400 and one or more LANs 422 and/or WANs.

The network link/s 420 typically provides data communication through one or more networks to other data devices. For example, the network link/s 420 may provide a connection through one or more local area networks 422 (LANs) to one or more host computers 424 or to data equipment operated by an Internet Service Provider (ISP) 426. The ISP 426 provides connectivity to one or more wide area networks 428, such as the Internet. The LAN/s 422 and WAN/s 428 use electrical, electromagnetic, or optical signals that carry digital data streams. The signals through the various networks and the signals on the network link/s 420 and through the communication interface/s 418 are example forms of transmission media, or transitory media.

The term “storage media” as used herein refers to any non-transitory media that stores data and/or instructions that cause a machine to operate in a specific fashion. Such storage media may include volatile and/or non-volatile media. Storage media is distinct from but may be used in conjunction with transmission media. Transmission media participates in transferring information between storage media. For example, transmission media includes coaxial cables, copper wire and fiber optics, including traces and/or other physical electrically conductive components that comprise the bus 402. Transmission media can also take the form of acoustic or light waves, such as those generated during radio-wave and infra-red data communications.

Various forms of media may be involved in carrying one or more sequences of one or more instructions to the processor 404 for execution. For example, the instructions may initially be carried on a magnetic disk or solid-state drive of a remote computer. The remote computer can load the instructions into its main memory 406 and send the instructions over a telecommunications line using a modem. A modem local to the computer system 400 can receive the data on the telephone line and use an infra-red transmitter to convert the data to an infra-red signal. An infra-red detector can receive the data carried in the infra-red signal and appropriate circuitry can place the data on the bus 402. The bus 402 carries the data to main memory 406, from which the processor 404 retrieves and executes the instructions. The instructions received by main memory 406 may optionally be stored on the storage device 410 either before or after execution by the processor 404.

The computer system 400 can send messages and receive data, including program code, through the network(s), the network link 420, and the communication interface/s 418. In the Internet example, one or more servers 430 may transmit signals corresponding to data or instructions requested for an application program executed by the computer system 400 through the Internet 428, ISP 426, local network 422 and a communication interface 418. The received signals may include instructions and/or information for execution and/or processing by the processor/s 404. The processor/s 404 may execute and/or process the instructions and/or information upon receiving the signals by accessing main memory 406, or at a later time by storing them and then accessing them from the storage device/s 410.

Although the concepts herein have been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A system for occluding a left atrial appendage (“LAA”) of a patient, the system comprising:
a delivery device including a catheter and a delivery cable configured to be received within the catheter, the delivery cable including a distal connector, the distal connector including a first distal connector electrode, a first delivery device conducting wire having a distal end coupled to the first distal connector electrode and having a proximal end configured to couple to a navigation system external to the patient; and
a collapsible and expandable LAA occluder having a first implant electrode, the first implant electrode being positioned on the LAA occluder so that, when the LAA occluder is in an expanded condition within the LAA of the patient, the first implant electrode is in contact with tissue of the LAA, the LAA occluder including a proximal connector, the proximal connector including a first proximal connector electrode, a first implant conducting wire having a distal end coupled to the first implant electrode and having a proximal end coupled to the first proximal connector electrode;
wherein the system has (i) a delivery condition in which the distal connector is connected to the proximal connector to couple the LAA occluder to the delivery cable so that the first distal connector electrode is in contact with the first proximal connector electrode to conductively couple the first implant electrode with the proximal end of the delivery device conducting wire via the first implant conducting wire, and (ii) an implanted condition in which the distal connector is disconnected from the proximal connector to decouple the LAA occluder from the delivery cable, so that the first distal connector electrode is not in contact with the first proximal connector electrode.

2. The system of claim 1, wherein, in the expanded condition of the LAA occluder, the LAA occluder includes a distal lobe having a distal diameter, a proximal disc having a proximal diameter, and a waist connecting the proximal disc to the distal lobe, the waist having a waist diameter, the proximal diameter being larger than the distal diameter, the distal diameter being greater than the waist diameter.

3. The system of claim 2, wherein the proximal connector includes a tube portion, the tube portion extending into an interior of the disc and at least partially into an interior of the waist.

4. The system of claim 1, where the distal connector has threads, and the proximal connector has threads that are complementary to the threads of the distal connector.

5. The system of claim 4, wherein the system is configured to transition from the delivery condition to the implanted condition via rotation of the distal connector relative to the proximal connector.

6. The system of claim 3, wherein the system is configured to transition from the delivery condition to the implanted condition via pulling the distal connector proximally relative to the proximal connector.

7. The system of claim 6, wherein the first distal connector electrode is retractable at least partially into an interior of the distal connector.

8. The system of claim 7, wherein the first distal connector electrode is configured to retract at least partially into the interior of the distal connector by applying a proximal force on the first delivery device conducting wire.

9. The system of claim 1, wherein the distal connector includes an interior lumen, and the delivery cable includes an interior lumen, the first delivery device conducting wire extending through the interior lumen of the distal connector and through the interior lumen of the delivery cable.

10. The system of claim 1, further comprising the navigation system, wherein in the delivery condition of the system, the navigation system is configured to receive signals from the first implant electrode via the first implant conducting wire and via the first delivery device conducting wire.

11. The system of claim 1, wherein (i) the first distal connector electrode is one of a plurality of distal connector electrodes, (ii) the first delivery device conducting wire is one of a plurality of distal conducting wires, each of the plurality of distal conducting wires being coupled to a respective one of the plurality of distal connector electrodes, (iii) the first implant electrode is one of a plurality of implant electrodes, (iv) the first proximal connector electrode is one of a plurality of proximal connector electrodes, and (v) the first implant conducting wire is one of a plurality of implant conducting wires, each of the plurality of implant electrodes being coupled to a respective one of the plurality of proximal connector electrodes via a respective one of the plurality of implant conducting wires.

12. The system of claim 11, wherein the plurality of distal connector electrodes are arranged in a linear array, and the plurality of proximal electrode connectors are arranged in a linear array.

13. The system of claim 11, wherein the plurality of distal connector electrodes are arranged in a helical array, and the plurality of proximal electrode connectors are arranged in a helical array.

14. The system of claim 11, wherein the plurality of implant electrodes includes (i) an indicator electrode configured to contact tissue when the LAA occluder is in the expanded condition within the LAA of the patient, the first implant electrode being the indicator electrode, and (ii) a reference electrode configured to not contact tissue when the LAA occluder is in the expanded condition within the LAA of the patient.

15. The system of claim 11, wherein the plurality of implant electrodes includes a second implant electrode, the first implant electrode being positioned on a proximal disc of the LAA occluder, the second implant electrode being positioned on a distal lobe of the LAA occluder.

16. A method of occluding a left atrial appendage (“LAA”) of a patient, the method comprising:
advancing a LAA occluder through a catheter of a delivery device while the LAA occluder is in a collapsed condition and while a proximal connector of the LAA occluder is connected to a distal connector of a delivery cable of the delivery device in a delivery configuration;
allowing the LAA occluder to expand within the LAA of the patient so that a first implant electrode on the LAA occluder contacts tissue of the LAA while the proximal connector remains connected to the distal connector;
receiving a signal transmitted from the first implant electrode to a navigation system external to the patient when the first implant electrode is in contact with tissue of the LAA, the signal being transmitted (i) from the first implant electrode to a first proximal connector electrode on the proximal connector via a first implant conducting wire having a distal end coupled to the first implant electrode and having a proximal end coupled to the first proximal connector electrode, (ii) from the first proximal connector electrode to a first distal connector electrode on the distal connector, and (iii) from the first distal connector electrode to the navigation system via a first delivery device conducting wire having a distal end coupled to the first distal connector electrode and having a proximal end coupled to the navigation system; and
after receiving the signal, disconnecting the distal connector from the proximal connector so that the LAA occluder disconnects from the delivery device and so that the first distal connector electrode is no longer in contact with the first proximal connector electrode.

17. The method of claim 16, further comprising, after receiving the signal but before disconnecting the distal connector from the proximal connector, evaluating contact between the LAA occluder and the tissue of the LAA based on the signal received by the navigation system.

18. The method of claim 16, wherein (i) the first distal connector electrode is one of a plurality of distal connector electrodes, (ii) the first delivery device conducting wire is one of a plurality of distal conducting wires, each of the plurality of distal conducting wires being coupled to a respective one of the plurality of distal connector electrodes, (iii) the first implant electrode is one of a plurality of implant electrodes, (iv) the first proximal connector electrode is one of a plurality of proximal connector electrodes, and (v) the first implant conducting wire being is of a plurality of implant conducting wires, each of the plurality of implant electrodes being coupled to a respective one of the plurality of proximal connector electrodes via a respective one of the plurality of implant conducting wires.

19. The method of claim 18, wherein, upon allowing the LAA occluder to expand within the LAA of the patient, (i) the first implant electrode is on a distal lobe of the LAA occluder and contacts tissue of an interior wall of the LAA, and (ii) a second implant electrode of the plurality of implant electrodes is on a proximal disc of the LAA occluder and contacts tissue forming an ostium leading into the LAA.

20. The method of claim 19, wherein, upon allowing the LAA occluder to expand within the LAA of the patient, a third implant electrode of the plurality of implant electrodes does not contact tissue of the LAA, the third implant electrode being a reference electrode, the first and second implant electrodes being indicator electrodes.
